(19) United States
(12) Patent Application Publication
(10) Pub. No.: US 2021/0047651 A1
(43) Pub. Date: Feb. 18, 2021

(54) COMPOSITIONS AND METHODS FOR TARGETED INSERTION OF SELECTABLE MARKER-FREE DNA SEQUENCE IN PLANTS

(71) Applicants: Pamela C. RONALD, Davis, CA (US); Xiaoou DONG, Davis, CA (US)

(72) Inventors: Pamela C. RONALD, Davis, CA (US); Xiaoou DONG, Davis, CA (US)

(73) Assignee: The Regents of the University of California, Oakland, CA (US)

(21) Appl. No.: 16/927,385

(22) Filed: Jul. 13, 2020

Related U.S. Application Data
(60) Provisional application No. 62/873,691, filed on Jul. 12, 2019.

(51) Int. Cl. C12N 15/82

(52) U.S. Cl. CPC C12N 15/8201

(57) ABSTRACT

The present invention provides for a method for inserting a selectable marker-free DNA fragment into a target location in a genome of a plant cell.

### CROSS REFERENCE TO RELATED APPLICATIONS

The application claims priority to U.S. Provisional Patent Application Ser. No. 62/873,691, filed Jul. 12, 2019; which is incorporated herein by reference.

### STATEMENT OF GOVERNMENTAL SUPPORT

The invention was made with government support under Contract Nos. DE-AC02-05CH11231 awarded by the U.S. Department of Energy. The government has certain rights in the invention.

### FIELD OF THE INVENTION

The present invention is in the field of targeted gene insertion in plants.

### BACKGROUND OF THE INVENTION

Conventional Agrobacterium- or particle bombardment-based plant transformation integrates transgenes at random locations in the plant genome, which can sometimes reduce yield in the resulting plants¹. Genomic safe harbors (GSHs)² are chromosomal regions that can accommodate transgenes without adverse effects on the host organism due to genome disruption. Targeted gene insertion at double-strand breaks (DSBs) in the GSHs provides a desirable alternative to conventional plant transformation methods³. Recent advances in genome editing technologies have enabled the induction of DSB at defined targets in a relatively simple manner, paving the way for targeted gene insertion in plants^{4,5}.

CRISPR-Cas is by far the most widely-used genome editing platform due to its efficacy, versatility, and simplicity⁶. The CRISPR-Cas system typically consists of a sequence-specific nuclease such as Cas9, and a guide RNA (gRNA), which mediates the recognition of a target sequence and cleavage at that site by the nuclease. Although efficient CRISPR-Cas-based tools for gene knockout in diverse plant species have been developed⁷⁻¹⁰, targeted gene insertion in plants by CRISPR-Cas has proved to be more challenging^{11,12}. Most reported examples of targeted gene insertion by CRISPR-Cas in plants are dependent on chemical selection of the inserted cassette¹³⁻¹⁹. For this reason, a selectable marker gene is often included in the inserted cassette to enable selection with an herbicide. A disadvantage to this approach is that the marker gene takes up valuable space within the cassette and it is retained in subsequent generations together with the desired trait. Products obtained through such approaches often require additional regulatory approvals and can trigger public concern. The few cases of targeted insertion of marker-free DNA fragments in plants have been achieved with relatively small DNA fragments (ranging from 281 bp to 1.8 kb)²⁰⁻²³. The small size of the DNA insert restricts the amount of genetic information that can be introduced.

Current methods of targeted insertion of selectable marker-free DNA in plants are disclosed in the following:
1. Li, J., et al., Gene replacements and insertions in rice by intron targeting using CRISPR-Cas9. Nat Plants, 2016. 2: p. 16139.
2. Lu, Y., et al. Targeted, efficient sequence insertion and replacement in rice. Nat Biotechnol, 2020.
3. Dahan-Meir, T., et al. Efficient in planta gene targeting in tomato using geminiviral replicons and the CRISPR/Cas9 system. Plant J, 2018. 95(1): p. 5-16.
4. Shi, J., et al. ARGOS8 variants generated by CRISPR-Cas9 improve maize grain yield under field drought stress conditions. Plant Biotechnol J. 2017. 15(2): p. 207-216.

### SUMMARY OF THE INVENTION

The present invention provides for a method for inserting a selectable marker-free DNA fragment into a target location in a genome of a plant cell.

The present invention provides for a method for inserting a DNA fragment into a target location in a genome of a plant cell, said method comprising: (a) providing a composition comprising an first nucleic acid encoding a cassette comprising one or more genes of interest (GOI) flanked by a left arm 5′ of the cassette and a right arm 3′ of the cassette, and a first guide RNA target sequence 5′ of the left arm and a second guide RNA target sequence 3′ of the right arm, and a second nucleic acid encoding a Cas9p, a guide RNA, and a selectable marker; (b) introducing the first nucleic acid and the second nucleic acid into a plant cell, and (c) growing the plant cell on a medium that selects for the marker.

In some embodiments, the plant cells comprise a plant callus. In some embodiments, the method further comprises: (d) regenerating a seedling from a plant callus such that the seedling grows into a plant. In some embodiments, the method further comprises: (e) confirming the insertion of the GOI in a targeted location of the genome of the plant regenerating seedlings from the plant calli.

The present invention provides for a composition comprising an isolated first nucleic acid encoding a cassette comprising one or more genes of interest (GOI) flanked by a left arm 5′ of the cassette and a right arm 3′ of the cassette, and a first guide RNA target sequence 5′ of the left arm and a second guide RNA target sequence 3′ of the right arm. In some embodiments, the GOI encodes one or more enzymes. In some embodiments, the one or more enzymes are one or more biosynthetic enzymes. In some embodiments, the one or more biosynthetic enzymes are from a biosynthetic pathway for producing a compound. In a particular embodiment, the compound is a carotenoid.

In some embodiments, the composition further comprises an isolated second nucleic acid encoding a Cas9p, a guide RNA, and a marker. In some embodiments, the marker is an antibiotic or herbicide resistance marker.

In some embodiments, the first nucleic acid and/or the second nucleic acid is a vector or plasmid.

In some embodiments, the plant cell, calli or plant is a monocot. In some embodiments, the monocot is a grass. In some embodiments, the grass is a rice.

The present invention provides for an optimized method to insert large, marker-free DNA fragments at designated genomic targets in a plant, such as a grass, such as a rice. The method comprises the cleavage of genomic DNA in rice cells at a specific target using CRISPR-Cas, followed by the incorporation of the exogenously supplied donor DNA at the cleavage site. In some embodiments, the CRISPR-Cas reagents and the donor DNA are encoded on two separate plasmids, which are delivered together into plant cells using a particle gun. Plants with the intended insertion can be identified from the regenerated population by PCR screening. The method is capable of targeted insertion of a biosynthesis cassette, such as a carotenoid biosynthesis cassette with two genes crtI and psy, at a pre-determined genomic safe harbor in rice. The method is capable of inserting a DNA fragment, such as a marker-free DNA fragment, having a size equal to or larger than about 3.4 kb, about 3.5 kb, about 3.6 kb, about 3.7 kb, about 3.8 kb, about 4.0 kb, about 4.5 kb, about 5.0 kb, or about 5.2 kb.

### BRIEF DESCRIPTION OF THE DRAWINGS

The foregoing aspects and others will be readily appreciated by the skilled artisan from the following description of illustrative embodiments when read in conjunction with the accompanying drawings.

FIG. 1A. Scheme for targeted insertion of the carotenoid cassette. Map of the donor plasmid pAcc-B with details of the carotenoid cassette (orange arrow). Red and blue arrows represent the homology arms. The two vertical green triangles mark the positions of the guide RNA B target sites. The nucleotide sequence of the donor plasmid is provided. Primers used to genotype the donor plasmid are marked on the map.

FIG. 1B. Scheme for targeted insertion of the carotenoid cassette. Map of the CRISPR plasmid pCam1300-CRISPR-B. Genes encoding Cas9p, gRNA-B, and hygromycin resistance (Hyg^{R}) are represented by purple, green, and black arrows, respectively. The Cas9p module is shown in detail. Primers used to genotype Cas9p are marked on the map. c, Scheme for transformation, selection, and regeneration.

FIG. 1C. Scheme for targeted insertion of the carotenoid cassette. Scheme for transformation, selection, and regeneration.

FIG. 2A. Molecular characterization of the carotenoid cassette at Target B. Diagram of the inserted carotenoid cassette at Target B in T0 plants #11, 16, 17, 24, 28, 48, and 50. The junction sequences in all the seven plants are identical, as shown in the diagram. For convenience, only the sequencing chromatograms for T0 #48 are shown. The protospacer adjacent motif (PAM) of the original guide RNA B targets are highlighted in yellow.

FIG. 2B. Molecular characterization of the carotenoid cassette at Target B. Diagram of the inserted carotenoid cassette at Target B in T0 plants #11, 16, 17, 24, 28, 48, and 50. The junction sequences in all the seven plants are identical, as shown in the diagram. For convenience, only the sequencing chromatograms for T0 #48 are shown. The protospacer adjacent motif (PAM) of the original guide RNA B targets are highlighted in yellow. The nucleotide sequences are SEQ ID NO:70 and SEQ ID NO:71, respectively.

FIG. 3. Genetic segregation of the progeny of T0-48A. a, Genotyping the T1 progeny of T0-48A. The purpose of each PCR experiment and the genotyping primers used are shown to the left for each gel panel. Primers Cas9p-Genotyping-F and nos-Terminator-R amplify a 534 bp DNA fragment in plants with the Cas9 module. b, Genotyping the T1 progeny of T0-48A. The purpose of each PCR experiment and the genotyping primers used are shown to the left for each gel panel. Primers M13F and 1R amplify a 1.8 kb DNA fragment in plants with the off-target insertion of the pAcc-B donor plasmid. c, Genotyping the T1 progeny of T0-48A. The purpose of each PCR experiment and the genotyping primers used are shown to the left for each gel panel. Primers 1F and 2F amplify a 2.3 kb DNA fragment in plants with the carotenoid cassette inserted at Target B. d, Genotyping the T1 progeny of T0-48A. The purpose of each PCR experiment and the genotyping primers used are shown to the left for each gel panel. Primers 1F and 3R amplify a 1.9 kb genomic DNA fragment in plants unless the carotenoid cassette at Target B is homozygous. The positions of the primers used are illustrated in FIGS. 1A, 1B, and FIG. 2A. Kitaake (K) is used as the negative control. The red triangle at the bottom marks 48A-7, which is free of the co-integrated CRISPR and donor plasmids.

FIG. 4A. Trait assessment of the homozygous carotenoid-enriched rice line 48A-7. Morphology of the 70-day-old Kitaake and 48A-7 plants.

FIG. 4B. Trait assessment of the homozygous carotenoid-enriched rice line 48A-7. Grain length comparison between Kitaake and the progeny of 48A-7.

FIG. 4C. Trait assessment of the homozygous carotenoid-enriched rice line 48A-7. Grain width comparison between Kitaake and the progeny of 48A-7.

FIG. 4D. Trait assessment of the homozygous carotenoid-enriched rice line 48A-7. Dry grain weight of randomly picked seeds from Kitaake and the progeny of 48A-7 (n=100). Horizontal bars represent the mean value.

FIG. 4E. Trait assessment of the homozygous carotenoid-enriched rice line 48A-7. Picture of 100 randomly picked dehusked seeds from Kitaake and the progeny of 48A-7. White scale bars represent 1 cm.

FIG. 5A. Removal of the 2.4 kb plasmid fragment from 48A-7 by backcross. Checking the homozygosity of the carotenoid cassette inserted at Target B in the F2 individuals 1-11 and 2-8 from the backcross between 48A-7 and Kitaake. PCR primers 1F and 3R anneal to genomic positions flanking Target B, as shown in FIG. 2A, amplifying a 1.9 kb genomic DNA fragment unless the carotenoid cassette at Target B is homozygous. Kitaake and 48A-7 are used as the wild type and homozygous controls, respectively.

FIG. 5B. Removal of the 2.4 kb plasmid fragment from 48A-7 by backcross. Detecting the presence of the 2.4 kb CRISPR plasmid fragment on Chromosome 5 by PCR. Primers Chr5-insert-flanking-L and Chr5-insert-flanking-R amplify a 446 bp DNA fragment when the plasmid fragment is absent, or a 2.8 kb DNA fragment when the plasmid fragment is present. Kitaake and 48A-7 are used as the wild type and homozygous controls, respectively.

FIG. 5C. Removal of the 2.4 kb plasmid fragment from 48A-7 by backcross. Picture of 100 randomly picked dehusked seeds from Kitaake and the two F2 plants described in FIG. 5A.

FIG. 6. Verifying homozygous mutations in the five rice mutants. Homozygous mutations listed in Table 1 are verified by PCR in the corresponding rice mutant lines. Each panel shows the genotyping of one mutation site. The primers used are indicated below each panel. The first four panels show the genotyping of the four fast-neutron (FN) mutant lines in Table 1. For each mutant line, four siblings are included as biological replicates. KitaakeX (X), the genetic background of these FN mutants, is included as the negative control. The last panel shows the genotyping of two KitaakeX plants at the homozygous XA21 transgene insertion site. Kitaake (K), the genetic background of KitaakeX (X), is included as the negative control.

TABLE 1
Five fast-neutron rice mutants with wild type morphology
and the homozygous mutations they carry.

| Mutant | Genetic | Type of mutation | | Name of candidate |
|---|---|---|---|---|
| name | background | (homozygous) | Genomic position | genomic safe harbor |
| FN 226 | KitaakeX | Insertion | Chr6:12611821 | GSH-A |
| FN 494 | KitaakeX | Translocation | Chr1:42355239 | GSH-B |
| FN 497 | KitaakeX | Insertion | Chr1:4898432 | GSH-C |
| FN 867 | KitaakeX | Insertion | Chr3:10490206 | GSH-D |
| KitaakeX | Kitaake | Transgene insertion | Chr6:28154158 | GSH-E |

Note: Genomic positions of the mutations are based on the KitaakeX reference genome. Detailed information on the mutations can be found at the webpage for: kitbase.ucdavis.edu. All plants in the first column exhibit Kitaake wild type-like morphology.

FIG. 7A. Identifying guide RNA targets where Cas9 cleavages efficiently. Nucleotide sequences of the seven guide RNA targets. The protospacer adjacent motif (PAM) sequences are colored in red. The nucleotide sequences are SEQ ID NO:72 to SEQ ID NO:78, respectively.

FIG. 7B. Identifying guide RNA targets where Cas9 cleavages efficiently. T7 Endonuclease I (T7E1) assay performed using rice protoplasts transiently expressing Cas9 and each of the guide RNAs shown in FIG. 7A. Protoplasts transformed with the empty expression vector pAHC17 without the Cas9-gRNA module are used as the negative (N) control for each guide RNA tested. DNA template with and without a heterozygous single nucleotide variance was included as the positive (+) and negative (−) T7E1 digestion control, respectively. The numbers below the lanes indicate the proportion of DNA cleaved by T7E1.

FIG. 8A. The entire donor plasmid integrated at Target B in T0 plant #1. Genotyping the 55 T0 plants using primers 1F and 1R

FIG. 8B. The entire donor plasmid integrated at Target B in T0 plant #1. Genotyping T0 plant #1 using primers 1F and 1R. The asterisk (*) marks a hypothesized secondary amplification product due to a PCR artifact that eliminates one copy of the left arm.

FIG. 8C. The entire donor plasmid integrated at Target B in T0 plant #1. Genotyping T0 plant #1 using primers 2F and 3R. Kitaake (K) was used as the negative control in FIGS. 8A to 8C.

FIG. 8D. The entire donor plasmid integrated at Target B in T0 plant #1. Diagram of the inserted plasmid at Target B in T0 plant #1. The broken grey lines represent the scaffold of the donor plasmid pAcc-B. Primers and the corresponding PCR products are illustrated in the diagram. Sequences covering both junction ends are shown with the sequencing chromatograms displayed. The green triangles mark the joining positions. The protospacer adjacent motif (PAM) of the original guide RNA B targets are highlighted in yellow. The nucleotide sequences are SEQ ID NO:79 and SEQ ID NO:80, respectively.

FIG. 9A. The carotenoid cassette is inserted at Target B in seven T0 plants through end joining. Genotyping the 55 T0 plants using primers 1F and 2F

FIG. 9B. The carotenoid cassette is inserted at Target B in seven T0 plants through end joining. Genotyping the seven T0s plants that are positive in FIG. 9A for both junctions of the insert. A diagram illustrates the hypothesized insertion, the genotyping primers, and the expected PCR products. Gel pictures below the diagram are the genotyping results. Kitaake (K) is used as the negative control in FIGS. 9A and 9B.

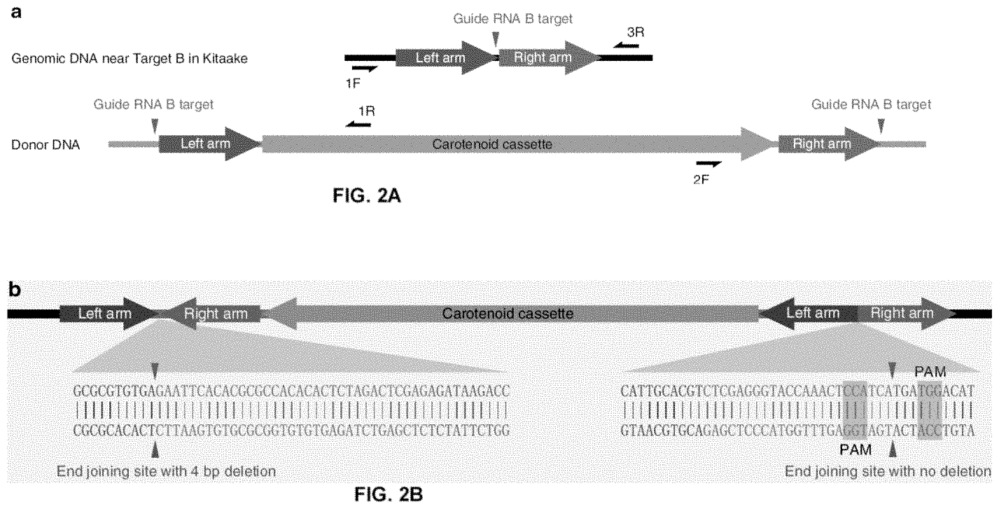

FIG. 10A. Homozygous insertion of the full-length carotenoid cassette at Target B in plant 48A-7. Genotyping 48A-7 for the full-length carotenoid cassette at Target B. PCR primers 1F and 3R anneal to genomic positions flanking Target B, as shown in FIG. 2A.

FIG. 10B. Homozygous insertion of the full-length carotenoid cassette at Target B in plant 48A-7. Electrophoresis of BamHI-digested genomic DNA from 48A-7 and Kitaake (K). Linearized donor plasmid pAcc-B is used as the control.

FIG. 10C. Homozygous insertion of the full-length carotenoid cassette at Target B in plant 48A-7. Southern Blot of the gel in (b). DNA ladders are shown on the right side while the sizes of the predicted hybridized fragments in 48A-7 are indicated by arrows on the left.

FIG. 10D. Homozygous insertion of the full-length carotenoid cassette at Target B in plant 48A-7. Diagram showing the probe used in FIG. 10C and the genomic fragments it hybridizes with in 48A-7 (upper) and Kitaake (lower).

FIG. 11A. Accumulation of β-carotene in seeds from 48A-7. High-performance liquid chromatography elution profiles of rice flour prepared from 48A-7 (middle) and Kitaake (lower) and a commercial β-carotene standard as reference (upper). The peak corresponding to (3-carotene is indicated by arrows. The x-axis is the retention time in minutes while the y-axis is the absorbance at 440 nm. mAU, milli absorbance unit.

FIG. 11B. Accumulation of β-carotene in seeds from 48A-7. Absorption spectra of the commercial β-carotene standard (upper) and the main peak of 48A-7 (lower), with the x-axis indicating wavelengths and the y-axis indicating absorbance.

FIG. 12. Presence of the CRISPR plasmid fragment in 48A-7 and multiple T0 plants. Detecting the insertion of the CRISPR plasmid fragment on Chromosome 5 by PCR. Primers Chr5-insert-flanking-L and Chr5-insert-flanking-R amplify a 446 bp DNA fragment when the plasmid fragment is absent, or a 2.8 kb DNA fragment when the plasmid fragment is present. Kitaake (K) is used as the negative control.

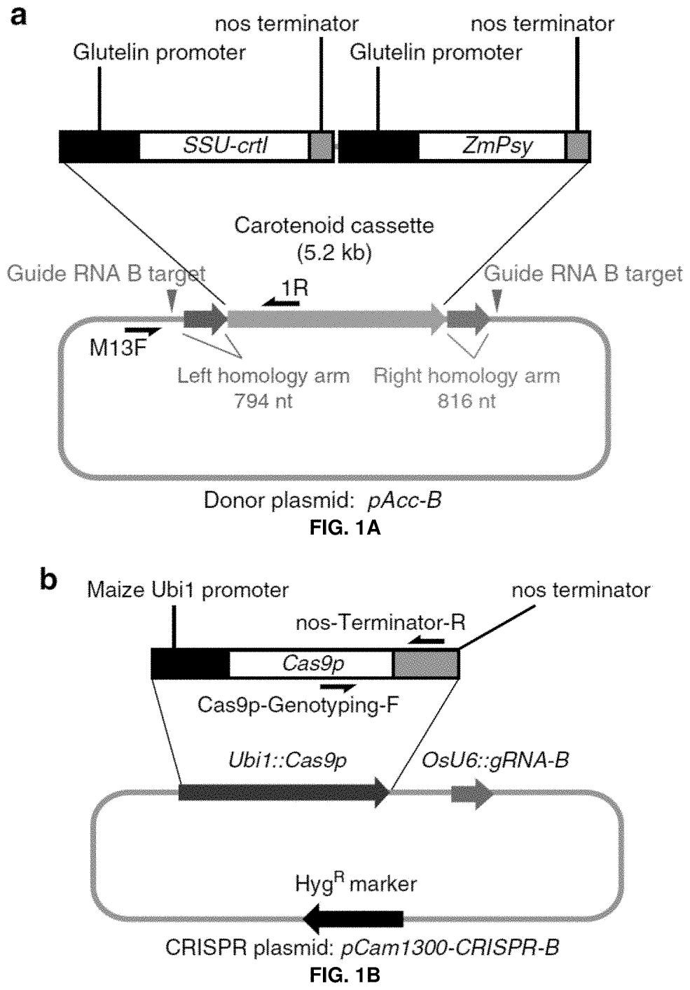

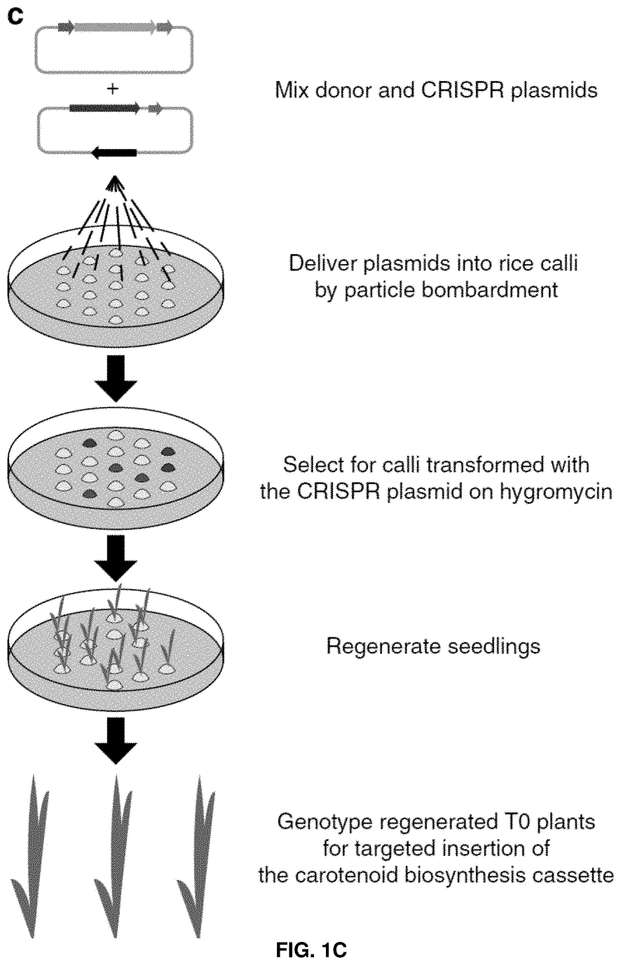

FIG. 13A. The golden seed color co-segregates with the presence of the carotenoid cassette. Genotyping T1 progenies derived from T0-48P, a tiller of T0 plant #48. The purpose of each PCR experiment and the genotyping primers used are shown to the left for each gel panel. From top to bottom: Primers Cas9p-Genotyping-F and nos-Terminator-R amplify a 534 bp DNA fragment in plants with the Cas9 module; Primers M13F and 1R amplify a 1.8 kb DNA fragment in plants with the off-target insertion of the pAcc-B donor plasmid; Primers 1F and 2F amplify a 2.3 kb DNA fragment in plants with the carotenoid cassette inserted at Target B; Primers 1F and 3R amplify a 1.9 kb genomic DNA fragment in plants unless the carotenoid cassette at Target B is homozygous. The positions of the primers used are illustrated in FIGS. 1A, 1B, and FIG. 2A. Kitaake (K) is used as the negative control. The red triangle marks 48P-3.

FIG. 13B. The golden seed color co-segregates with the presence of the carotenoid cassette. Genotyping progeny of the T1 individual 48P-3 for the presence of the carotenoid cassette at Target B. Plants #1-8 are derived from white seeds while plants #9-16 are derived from yellow seeds. Kitaake (K) is used as the negative control.

FIG. 14A. Maps of plasmids used for targeted gene insertion at Target C. Map of the donor plasmid pAcc-C with details of the carotenoid cassette (orange arrow). Brown and green arrows represent the homology arms. The two vertical red triangles mark the two guide RNA C target sites. The nucleotide sequence of the donor plasmid is provided.

FIG. 14B. Maps of plasmids used for targeted gene insertion at Target C. Map of the CRISPR plasmid pCam1300-CRISPR-C. Genes encoding Cas9p, gRNA-C, and hygromycin resistance (HygR) are represented by purple, red, and black arrows, respectively. The Cas9p module is shown in detail. Primers used to genotype Cas9p are marked on the map.

FIG. 15A. Insertion of the carotenoid cassette at Target C. Diagrams showing the genomic region near Target C in Kitaake rice and the donor DNA. Grey lines represent plasmid backbone DNA while black lines represent Kitaake genomic DNA. The vertical red triangles mark the positions of the guide RNA C targets.

FIG. 15B. Insertion of the carotenoid cassette at Target C. Genotyping T0 plant #6 for the insertion of the carotenoid cassette at Target C. A diagram illustrates the insertion, the genotyping primers, and the expected PCR products. Gel pictures below the diagram are the actual genotyping results. Kitaake (K) is used as the negative control.

FIG. 15C. Insertion of the carotenoid cassette at Target C. Junction border sequences of the carotenoid insert in T0 plant #6. Sanger sequencing chromatograms of the PCR products in FIG. 15B are shown. The protospacer adjacent motif (PAM) of the original guide RNA C targets are highlighted in yellow. The nucleotide sequences are SEQ ID NO:81 and SEQ ID NO:82, respectively.

FIG. 16. Scheme showing the on-target insertion of the cassette plus the arms, or the entire donor plasmid, through end-joining.

FIG. 17. Plasmid constructs shown for pAcc-B, the “Arms only B” plasmid, the “Cas only B” plasmid, and pAcc-C.

### DETAILED DESCRIPTION OF THE INVENTION

Before the invention is described in detail, it is to be understood that, unless otherwise indicated, this invention is not limited to particular sequences, expression vectors, enzymes, host microorganisms, or processes, as such may vary. It is also to be understood that the terminology used herein is for purposes of describing particular embodiments only, and is not intended to be limiting.

In this specification and in the claims that follow, reference will be made to a number of terms that shall be defined to have the following meanings:

The terms “optional” or “optionally” as used herein mean that the subsequently described feature or structure may or may not be present, or that the subsequently described event or circumstance may or may not occur, and that the description includes instances where a particular feature or structure is present and instances where the feature or structure is absent, or instances where the event or circumstance occurs and instances where it does not.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

The term “about” refers to a value including 10% more than the stated value and 10% less than the stated value.

As used herein, the term “promoter” refers to a polynucleotide sequence capable of driving transcription of a DNA sequence in a cell. Thus, promoters used in the polynucleotide constructs of the invention include cis- and trans-acting transcriptional control elements and regulatory sequences that are involved in regulating or modulating the timing and/or rate of transcription of a gene. For example, a promoter can be a cis-acting transcriptional control element, including an enhancer, a promoter, a transcription terminator, an origin of replication, a chromosomal integration sequence, 5′ and 3′ untranslated regions, or an intronic sequence, which are involved in transcriptional regulation. These cis-acting sequences typically interact with proteins or other biomolecules to carry out (turn on/off, regulate, modulate, etc.) gene transcription. Promoters are located 5′ to the transcribed gene, and as used herein, include the sequence 5′ from the translation start codon.

A “constitutive promoter” is one that is capable of initiating transcription in nearly all cell types, whereas a “cell type-specific promoter” initiates transcription only in one or a few particular cell types or groups of cells forming a tissue. In some embodiments, the promoter is secondary cell wall-specific and/or fiber cell-specific. A “fiber cell-specific promoter” refers to a promoter that initiates substantially higher levels of transcription in fiber cells as compared to other non-fiber cells of the plant. A “secondary cell wall-specific promoter” refers to a promoter that initiates substantially higher levels of transcription in cell types that have secondary cell walls, e.g., lignified tissues such as vessels and fibers, which may be found in wood and bark cells of a tree, as well as other parts of plants such as the leaf stalk. In some embodiments, a promoter is fiber cell-specific or secondary cell wall-specific if the transcription levels initiated by the promoter in fiber cells or secondary cell walls, respectively, are at least 3-fold, 4-fold, 5-fold, 6-fold, 7-fold, 8-fold, 9-fold, 10-fold, 50-fold, 100-fold, 500-fold, 000-fold higher or more as compared to the transcription levels initiated by the promoter in other tissues, resulting in the encoded protein substantially localized in plant cells that possess fiber cells or secondary cell wall, e.g., the stem of a plant. Non-limiting examples of fiber cell and/or secondary cell wall specific promoters include the promoters directing expression of the genes IRX1, IRX3, IRX5, IRX7, IRX8, IRX9, IRX10, IRX14, NST1, NST2, NST3, MYB46, MYB58, MYB63, MYB83, MYB85, MYB103, PAL1, PAL2, C3H, CcOAMT, CCR1, F5H, LAC4, LAC17, CADc, and CADd. See, e.g., Turner et al 1997; Meyer et al 1998; Jones et al 2001; Franke et al 2002; Ha et al 2002; Rohde et al 2004; Chen et al 2005; Stobout et al 2005; Brown et al 2005; Mitsuda et al 2005; Zhong et al 2006; Mitsuda et al 2007; Zhong et al 2007a, 2007b; Zhou et al 2009; Brown et al 2009; McCarthy et al 2009; Ko et al 2009; Wu et al 2010; Berthet et al 2011. In some embodiments, a promoter is substantially identical to a promoter from the lignin biosynthesis pathway. A promoter originated from one plant species may be used to direct gene expression in another plant species.

A polynucleotide or amino acid sequence is “heterologous” to an organism or a second polynucleotide or amino acid sequence if it originates from a foreign species, or, if from the same species, is modified from its original form. For example, when a polynucleotide encoding a polypeptide sequence is said to be operably linked to a heterologous promoter, it means that the polynucleotide coding sequence encoding the polypeptide is derived from one species whereas the promoter sequence is derived from another, different species; or, if both are derived from the same species, the coding sequence is not naturally associated with the promoter (e.g., is a genetically engineered coding sequence, e.g., from a different gene in the same species, or an allele from a different ecotype or variety, or a gene that is not naturally expressed in the target tissue).

The term “operably linked” refers to a functional relationship between two or more polynucleotide (e.g., DNA) segments. Typically, it refers to the functional relationship of a transcriptional regulatory sequence to a transcribed sequence. For example, a promoter or enhancer sequence is operably linked to a DNA or RNA sequence if it stimulates or modulates the transcription of the DNA or RNA sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance.

The terms “host cell” of “host organism” is used herein to refer to a living biological cell that can be transformed via insertion of an expression vector.

The terms “expression vector” or “vector” refer to a compound and/or composition that transduces, transforms, or infects a host cell, thereby causing the cell to express nucleic acids and/or proteins other than those native to the cell, or in a manner not native to the cell. An “expression vector” contains a sequence of nucleic acids (ordinarily RNA or DNA) to be expressed by the host cell. Optionally, the expression vector also comprises materials to aid in achieving entry of the nucleic acid into the host cell, such as a virus, liposome, protein coating, or the like. The expression vectors contemplated for use in the present invention include those into which a nucleic acid sequence can be inserted, along with any preferred or required operational elements. Further, the expression vector must be one that can be transferred into a host cell and replicated therein. Particular expression vectors are plasmids, particularly those with restriction sites that have been well documented and that contain the operational elements preferred or required for transcription of the nucleic acid sequence. Such plasmids, as well as other expression vectors, are well known to those of ordinary skill in the art.

The terms “polynucleotide” and “nucleic acid” are used interchangeably and refer to a single or double-stranded polymer of deoxyribonucleotide or ribonucleotide bases read from the 5′ to the 3′ end. A nucleic acid of the present invention will generally contain phosphodiester bonds, although in some cases, nucleic acid analogs may be used that may have alternate backbones, comprising, e.g., phosphoramidate, phosphorothioate, phosphorodithioate, or O-methylphophoroamidite linkages (see Eckstein, Oligonucleotides and Analogues: A Practical Approach, Oxford University Press); positive backbones; non-ionic backbones, and non-ribose backbones. Thus, nucleic acids or polynucleotides may also include modified nucleotides that permit correct read-through by a polymerase. “Polynucleotide sequence” or “nucleic acid sequence” includes both the sense and antisense strands of a nucleic acid as either individual single strands or in a duplex. As will be appreciated by those in the art, the depiction of a single strand also defines the sequence of the complementary strand; thus the sequences described herein also provide the complement of the sequence. Unless otherwise indicated, a particular nucleic acid sequence also implicitly encompasses variants thereof (e.g., degenerate codon substitutions) and complementary sequences, as well as the sequence explicitly indicated. The nucleic acid may be DNA, both genomic and cDNA, RNA or a hybrid, where the nucleic acid may contain combinations of deoxyribo- and ribo-nucleotides, and combinations of bases, including uracil, adenine, thymine, cytosine, guanine, inosine, xanthine hypoxanthine, isocytosine, isoguanine, etc.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

Several reports of targeted gene insertion in rice have been documented. Most of them are based on the CRISPR-Cas system. For example, Li et al. reported in 2016 the targeted insertion of a 1.6 kb DNA fragment at a designated genomic position in rice at a frequency of 2.2%. The method used did not rely on the selection of the insert, but the frequency of the insertion achieved is relatively low. Begemann et al. reported in 2017 the targeted insertion of a 3.3 kb DNA fragment in rice at a frequency of 8%. However, this method relies on the selection of the insertion events using herbicide, requiring the presence of a marker gene in the DNA to be inserted. The marker genes remain in the final product and often trigger additional regulations. Wang et al. reported in 2017 the targeted gene insertion of a 2.6 kb DNA fragment in rice with at a frequency of up to 8.5%. Similarly, this method requires that a selectable marker gene be included in the insert. Also, this method involves the integration of the replicon of a plant virus into the rice genome, which may trigger additional public concern.

Compared with the existing methods of targeted gene insertion in plants, the method has several advantages. Firstly, it does not rely on the use of selection to identify the insertion events. This means that a selectable marker gene is not required in the inserted DNA fragment. On one hand, this increases the capacity of the cassette to be inserted. On the other hand, the absence of selectable marker gene in the final product would potentially reduce the regulations involved. Secondly, the size of the DNA fragment inserted through this approach is larger than those achieved from the methods described previously. Thirdly, the method used does not involve the use of any plant viral replicon.

Reference cite (which are each incorporated by reference):
1 Kamthan, A., Chaudhuri, A., Kamthan, M. & Datta, A. Genetically modified (GM) crops: milestones and new advances in crop improvement. Theor Appl Genet 129, 1639-1655 (2016).
2 Papapetrou, E. P. & Schambach, A. Gene Insertion Into Genomic Safe Harbors for Human Gene Therapy. Mol Ther 24, 678-684 (2016).
3 Yamamoto, Y. & Gerbi, S. A. Making ends meet: targeted integration of DNA fragments by genome editing. Chromosoma 127, 405-420 (2018).
4 Sun, Y., Li, J. & Xia, L. Precise Genome Modification via Sequence-Specific Nucleases-Mediated Gene Targeting for Crop Improvement. Front Plant Sci 7, 1928 (2016).
5 Schindele, A., Dorn, A. & Puchta, H. CRISPR/Cas brings plant biology and breeding into the fast lane. Curr Opin Biotechnol 61, 7-14 (2019).
6 Barrangou, R. & Doudna, J. A. Applications of CRISPR technologies in research and beyond. Nat Biotechnol 34, 933-941 (2016).
7 Yin, K., Gao, C. & Qiu, J. L. Progress and prospects in plant genome editing. Nat Plants 3, 17107 (2017).
8 Mao, Y. et al. Application of the CRISPR-Cas system for efficient genome engineering in plants. Mol Plant 6, 2008-2011 (2013).
9 Miao, J. et al. Targeted mutagenesis in rice using CRISPR-Cas system. Cell Res 23, 1233-1236 (2013).
10 Shan, Q. W. et al. Targeted genome modification of crop plants using a CRISPR-Cas system. Nature Biotechnology 31, 686-688 (2013).
11 Collonnier, C. et al. Towards mastering CRISPR-induced gene knock-in in plants: Survey of key features and focus on the model Physcomitrella patens. Methods 121-122, 103-117 (2017).
12 Voytas, D. F. & Gao, C. Precision genome engineering and agriculture: opportunities and regulatory challenges. PLoS Biol 12, e1001877 (2014).
13 Li, Z. et al. Cas9-Guide RNA Directed Genome Editing in Soybean. Plant Physiol 169, 960-970 (2015).
14 Svitashev, S. et al. Targeted Mutagenesis, Precise Gene Editing, and Site-Specific Gene Insertion in Maize Using Cas9 and Guide RNA. Plant Physiol 169, 931-945 (2015).
15 Svitashev, S., Schwartz, C., Lenderts, B., Young, J. K. & Mark Cigan, A. Genome editing in maize directed by CRISPR-Cas9 ribonucleoprotein complexes. Nat Commun 7, 13274 (2016).
16 Begemann, M. B. et al. Precise insertion and guided editing of higher plant genomes using Cpf1 CRISPR nucleases. Sci Rep 7, 11606 (2017).
17 Wang, M. G. et al. Gene Targeting by Homology-Directed Repair in Rice Using a Geminivirus-Based CRISPR/Cas9 System. Mol Plant 10, 1007-1010 (2017).
18 Cermak, T., Baltes, N. J., Cegan, R., Zhang, Y. & Voytas, D. F. High-frequency, precise modification of the tomato genome. Genome Biol 16, 232 (2015).
19 Lee, K. et al. CRISPR/Cas9-mediated targeted T-DNA integration in rice. Plant Mol Biol 99, 317-328 (2019).
20 Shi, J. et al. ARGOS8 variants generated by CRISPR-Cas9 improve maize grain yield under field drought stress conditions. Plant Biotechnol J 15, 207-216 (2017).
21 Dahan-Meir, T. et al. Efficient in planta gene targeting in tomato using geminiviral replicons and the CRISPR/Cas9 system. Plant J (2018).
22 Miki, D., Zhang, W., Zeng, W., Feng, Z. & Zhu, J. K. CRISPR/Cas9-mediated gene targeting in Arabidopsis using sequential transformation. Nat Commun 9, 1967 (2018).
23 Li, J. et al. Gene replacements and insertions in rice by intron targeting using CRISPR-Cas9. Nat Plants 2, 16139 (2016).
24 Jung, K. H., An, G. & Ronald, P. C. Towards a better bowl of rice: assigning function to tens of thousands of rice genes. Nat Rev Genet 9, 91-101 (2008).
25 Li, G. et al. Genome-Wide Sequencing of 41 Rice (Oryza sativa L.) Mutated Lines Reveals Diverse Mutations Induced by Fast-Neutron Irradiation. Mol Plant 9, 1078-1081 (2016).
26 Li, G. et al. The Sequences of 1504 Mutants in the Model Rice Variety Kitaake Facilitate Rapid Functional Genomic Studies. Plant Cell 29, 1218-1231 (2017).
27 Xie, K., Zhang, J. & Yang, Y. Genome-wide prediction of highly specific guide RNA spacers for CRISPR-Cas9-mediated genome editing in model plants and major crops. Mol Plant 7, 923-926 (2014).
28 Jain, R. et al. Genome sequence of the model rice variety KitaakeX. BMC Genomics 20, 905 (2019).
29 Shan, Q., Wang, Y., Li, J. & Gao, C. Genome editing in rice and wheat using the CRISPR/Cas system. Nat Protoc 9, 2395-2410 (2014).
30 Paine, J. A. et al. Improving the nutritional value of Golden Rice through increased pro-vitamin A content. Nat Biotechnol 23, 482-487 (2005).
31 Ye, X. et al. Engineering the provitamin A (beta-carotene) biosynthetic pathway into (carotenoid-free) rice endosperm. Science 287, 303-305 (2000).
32 Swamy, B. P. M. et al. Compositional Analysis of Genetically Engineered GR2E “Golden Rice” in Comparison to That of Conventional Rice. J Agric Food Chem 67, 7986-7994 (2019).
33 Giuliano, G. Provitamin A biofortification of crop plants: a gold rush with many miners. Curr Opin Biotechnol 44, 169-180 (2017).
34 Engler, C., Kandzia, R. & Marillonnet, S. A one pot, one step, precision cloning method with high throughput capability. PLoS One 3, e3647 (2008).
35 Carroll, D. Genome engineering with targetable nucleases. Annu Rev Biochem 83, 409-439 (2014).
36 LePage, D. F. & Conlon, R. A. Animal models for disease: knockout, knock-in, and conditional mutant mice. Methods Mol Med 129, 41-67 (2006).
37 Song, F. & Stieger, K. Optimizing the DNA Donor Template for Homology-Directed Repair of Double-Strand Breaks. Mol Ther Nucleic Acids 7, 53-60 (2017).
38 Sun, Y. et al. Engineering Herbicide-Resistant Rice Plants through CRISPR/Cas9-Mediated Homologous Recombination of Acetolactate Synthase. Mol Plant 9, 628-631 (2016).
39 Yao, X. et al. Homology-mediated end joining-based targeted integration using CRISPR/Cas9. Cell Res 27, 801-814 (2017).
40 Ma, X. et al. A Robust CRISPR/Cas9 System for Convenient, High-Efficiency Multiplex Genome Editing in Monocot and Dicot Plants. Mol Plant 8, 1274-1284 (2015).
41 Fichtner, F., Urrea Castellanos, R. & Ulker, B. Precision genetic modifications: a new era in molecular biology and crop improvement. Planta 239, 921-939 (2014).
42 Chen, L. et al. Expression and inheritance of multiple transgenes in rice plants. Nat Biotechnol 16, 1060-1064 (1998).
43 Bae, S., Park, J. & Kim, J. S. Cas-OFFinder: a fast and versatile algorithm that searches for potential off-target sites of Cas9 RNA-guided endonucleases. Bioinformatics 30, 1473-1475 (2014).
44 Tang, X. et al. A large-scale whole-genome sequencing analysis reveals highly specific genome editing by both Cas9 and Cpf1 (Cas12a) nucleases in rice. Genome Biol 19, 84 (2018).
45 Schaub, P. et al. Nonenzymatic beta-Carotene Degradation in Provitamin A-Biofortified Crop Plants. J Agric Food Chem 65, 6588-6598 (2017).
46 Bechoff, A. et al. Effect of drying and storage on the degradation of total carotenoids in orange-fleshed sweetpotato cultivars. J Sci Food Agric 90, 622-629 (2010).
47 Bai, C. et al. Bottlenecks in carotenoid biosynthesis and accumulation in rice endosperm are influenced by the precursor-product balance. Plant Biotechnol J 14, 195-205 (2016).
48 Liu, J. et al. Genome-Scale Sequence Disruption Following Biolistic Transformation in Rice and Maize. Plant Cell 31, 368-383 (2019).
49 Bollinedi, H. et al. Molecular and Functional Characterization of GR2-R1 Event Based Backcross Derived Lines of Golden Rice in the Genetic Background of a Mega Rice Variety Swarna. PLoS One 12, e0169600 (2017).
50 Que, Q. et al. Trait stacking in transgenic crops: challenges and opportunities. GM Crops 1, 220-229 (2010).
51 Cobb, J. N., Biswas, P. S. & Platten, J. D. Back to the future: revisiting MAS as a tool for modern plant breeding. Theor Appl Genet 132, 647-667 (2019).
52 Leenay, R. T. et al. Large dataset enables prediction of repair after CRISPR-Cas9 editing in primary T cells. Nat Biotechnol 37, 1034-1037 (2019).
53 Lowder, L., Malzahn, A. & Qi, Y. Rapid Construction of Multiplexed CRISPR-Cas9 Systems for Plant Genome Editing. Methods Mol Biol 1578, 291-307 (2017).
54 Christensen, A. H., Sharrock, R. A. & Quail, P. H. Maize polyubiquitin genes: structure, thermal perturbation of expression and transcript splicing, and promoter activity following transfer to protoplasts by electroporation. Plant Mol Biol 18, 675-689 (1992).
55 Yoo, S. D., Cho, Y. H. & Sheen, J. Arabidopsis mesophyll protoplasts: a versatile cell system for transient gene expression analysis. Nat Protoc 2, 1565-1572 (2007).
56 Qin, X., Zhang, W., Dubcovsky, J. & Tian, L. Cloning and comparative analysis of carotenoid beta-hydroxylase genes provides new insights into carotenoid metabolism in tetraploid (Triticum turgidum ssp. durum) and hexaploid (Triticum aestivum) wheat grains. Plant Mol Biol 80, 631-646 (2012).

57 Li, H. & Durbin, R. Fast and accurate short read alignment with Burrows-Wheeler transform. Bioinformatics 25, 1754-1760 (2009).

58 Li, H. et al. The Sequence Alignment/Map format and SAMtools. Bioinformatics 25, 2078-2079 (2009).

59 Ye, K., Schulz, M. H., Long, Q., Apweiler, R. & Ning, Z. Pindel: a pattern growth approach to detect break points of large deletions and medium sized insertions from paired-end short reads. Bioinformatics 25, 2865-2871 (2009).

60 Chen, K. et al. BreakDancer: an algorithm for high-resolution mapping of genomic structural variation. Nat Methods 6, 677-681 (2009).

It is to be understood that, while the invention has been described in conjunction with the preferred specific embodiments thereof, the foregoing description is intended to illustrate and not limit the scope of the invention. Other aspects, advantages, and modifications within the scope of the invention will be apparent to those skilled in the art to which the invention pertains.

All patents, patent applications, and publications mentioned herein are hereby incorporated by reference in their entireties.

The invention having been described, the following examples are offered to illustrate the subject invention by way of illustration, not by way of limitation.

### Example 1

### Marker-Free Carotenoid-Enriched Rice Generated Through Targeted Gene Insertion Using CRISPR-Cas9

Targeted insertion of transgenes at pre-determined genomic safe harbors in plants provides a desirable alternative to transgene insertions at random sites through conventional methods. Most existing cases of targeted gene insertion in plants either relied on the presence of a selectable marker gene in the insertion cassette or occurred at low frequency with relatively small DNA fragments (<1.8 kb). Herein is a report of the use of an optimized CRISPR-Cas9-based method to achieve the targeted insertion of a 5.2 kb carotenoid biosynthesis cassette at two genomic safe harbors in rice. Marker-free rice plants are obtained with high carotenoid content in the seeds and no detectable penalty in morphology or yield. Whole-genome sequencing reveals the absence of off-target mutations by Cas9 in the engineered plants. These results demonstrate targeted gene insertion of marker-free DNA in rice using CRISPR-Cas9 genome editing and offer a promising strategy for genetic improvement of rice and other crops.

In this study, the targeted insertion of a 5.2 kb marker-free DNA fragment at two GSHs in rice using CRISPR-Cas is demonstrated, and homozygous carotenoid-enriched rice is obtained.

### Results

### Choosing Gene Insertion Targets in a Model Rice Variety

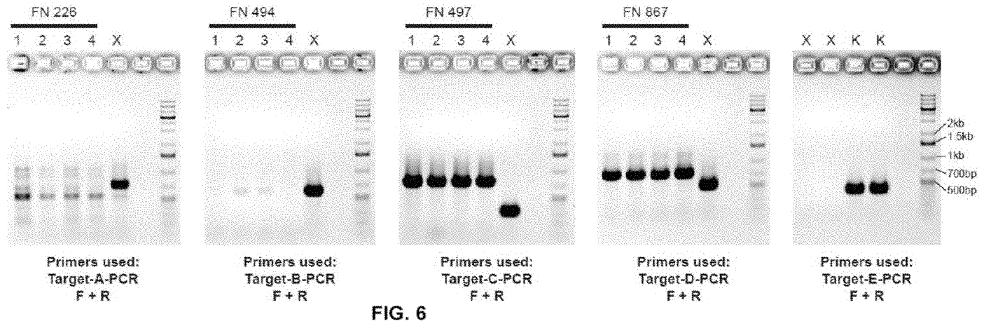

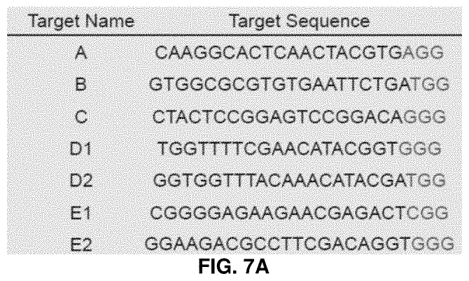

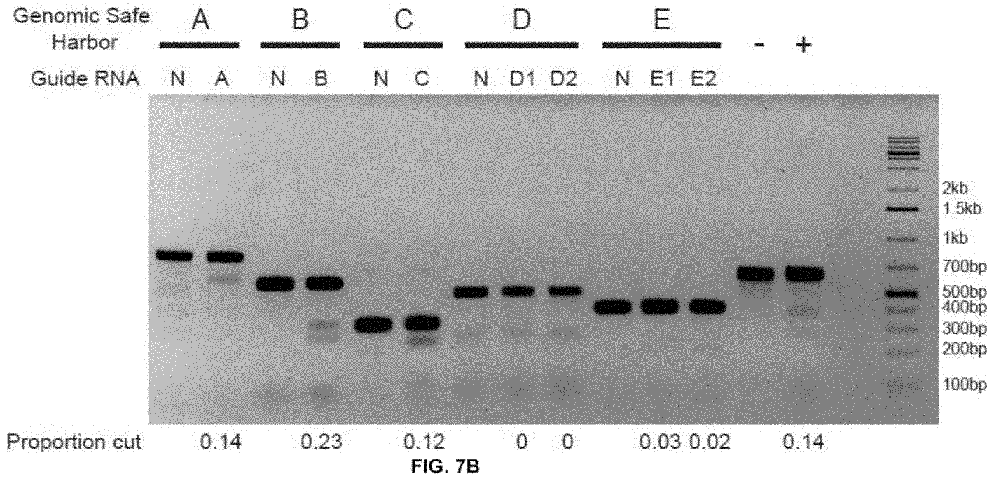

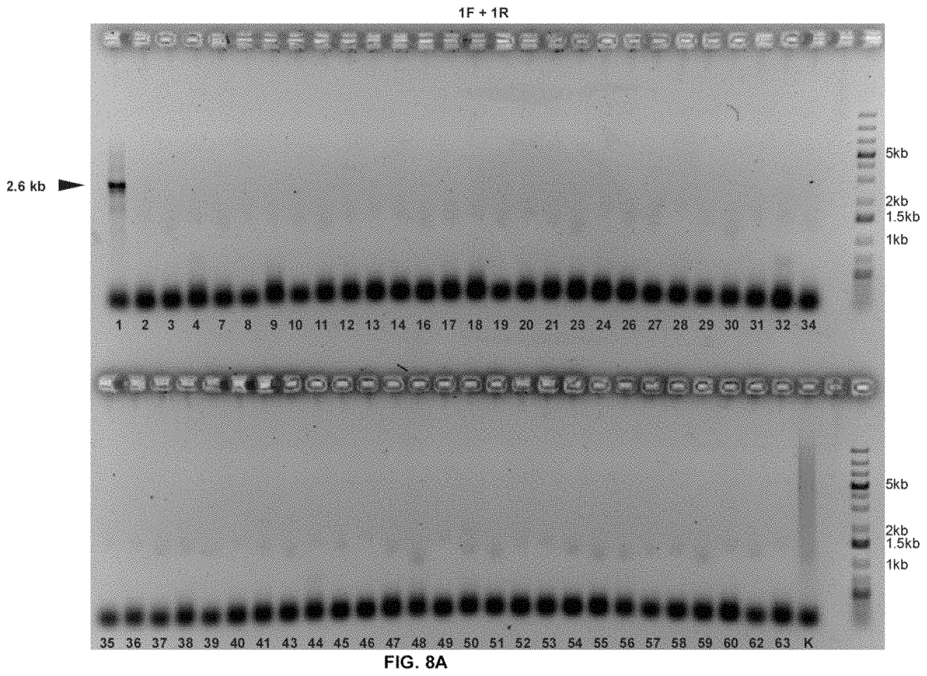

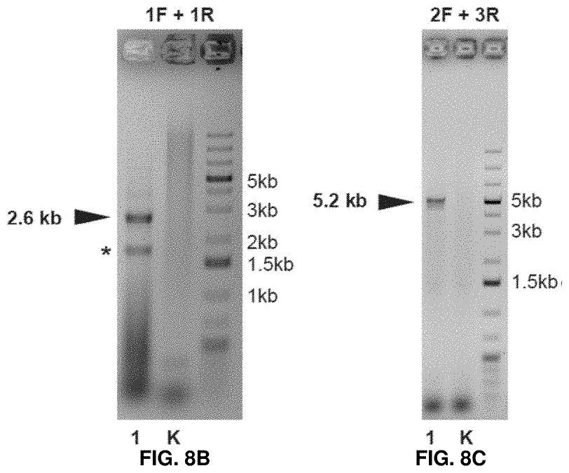

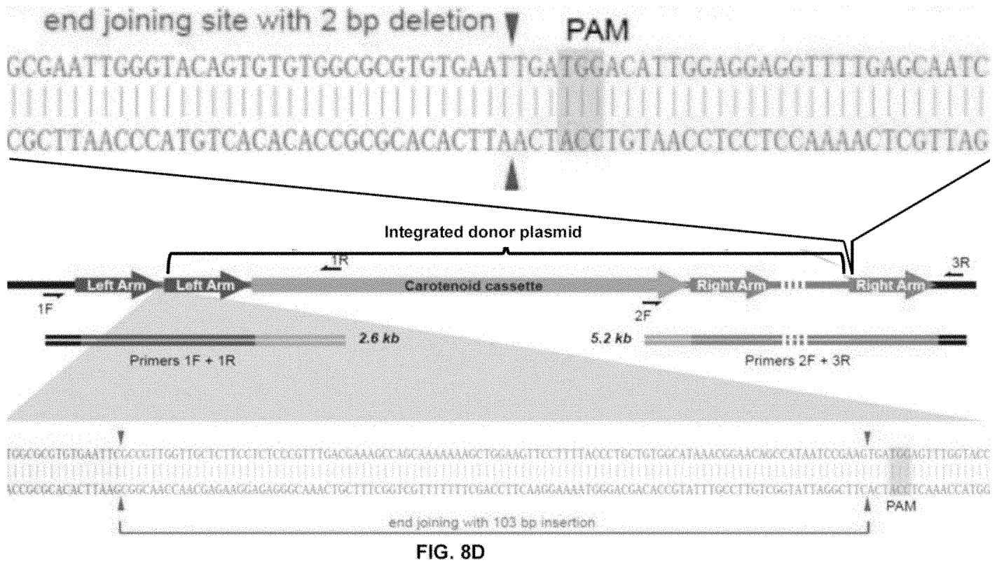

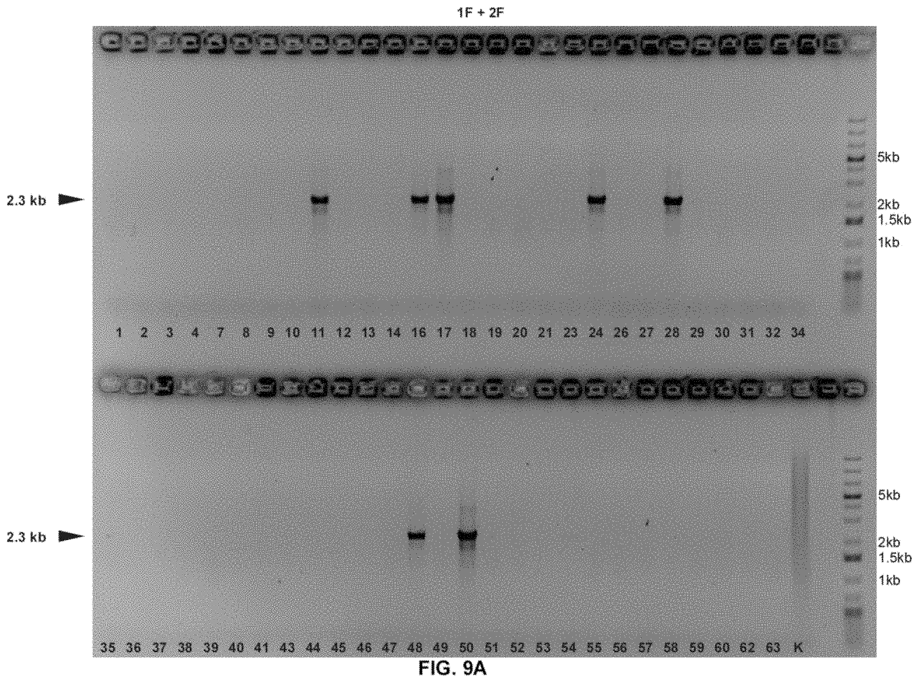

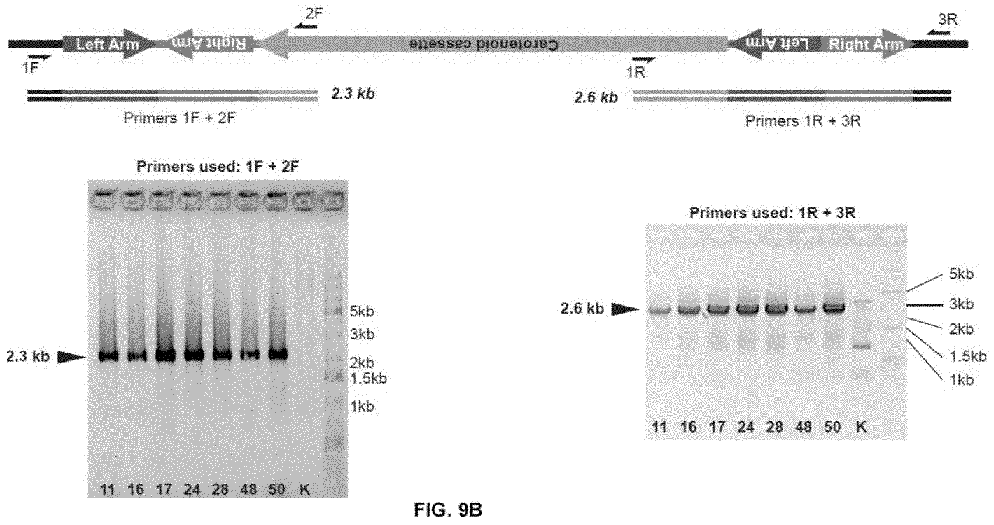

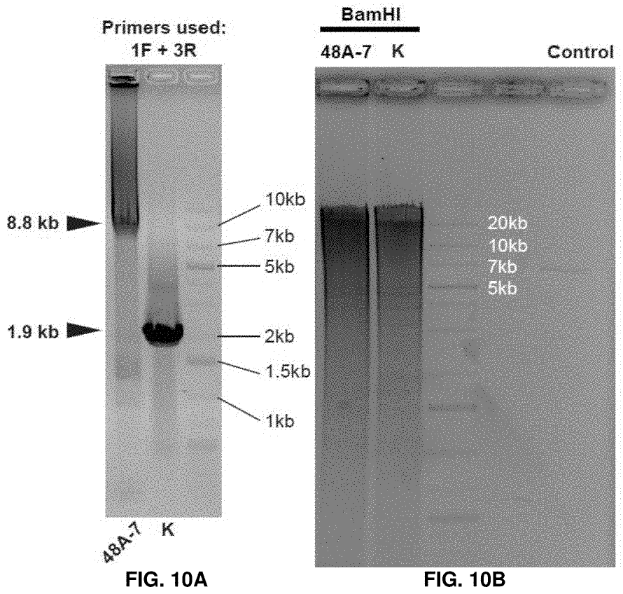

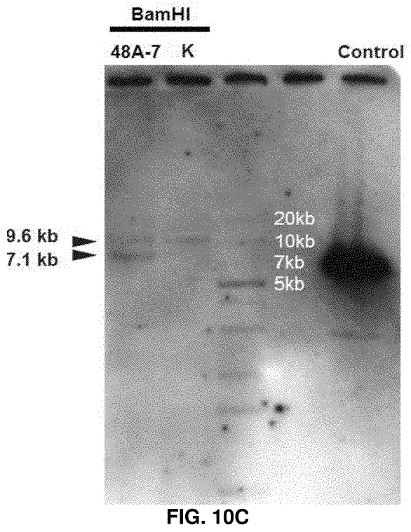

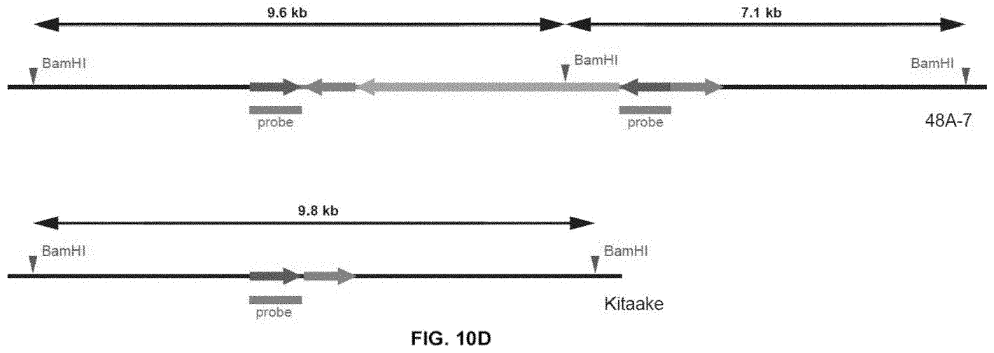

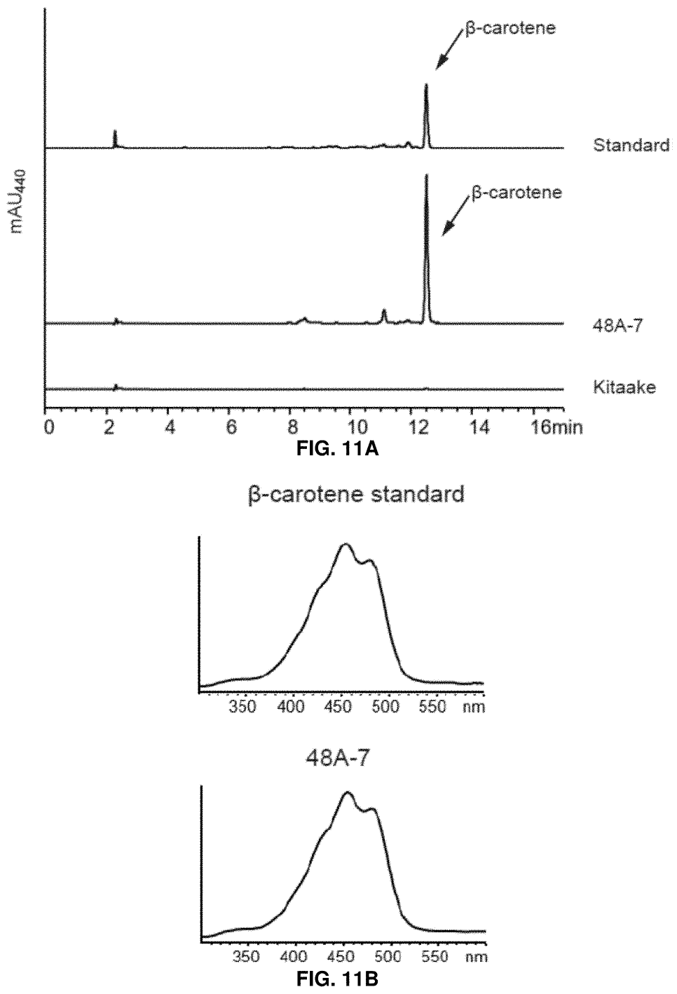

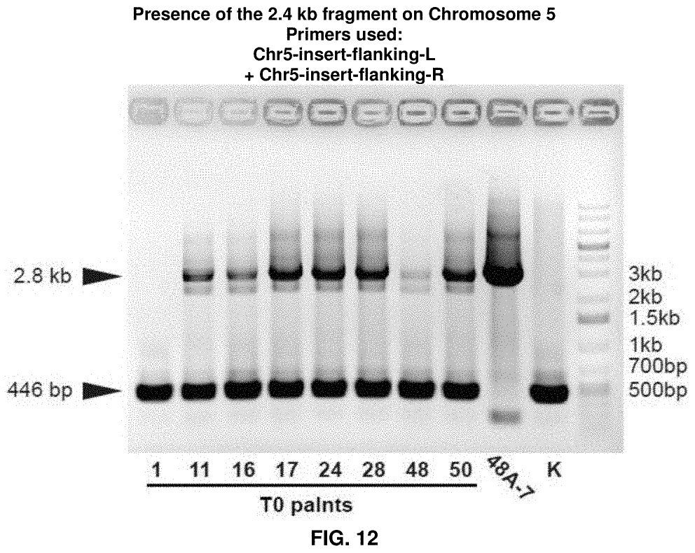

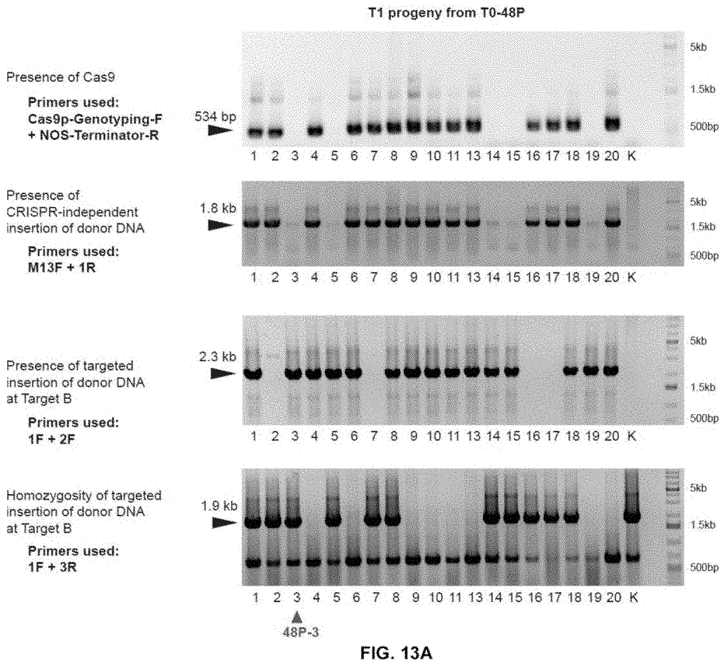

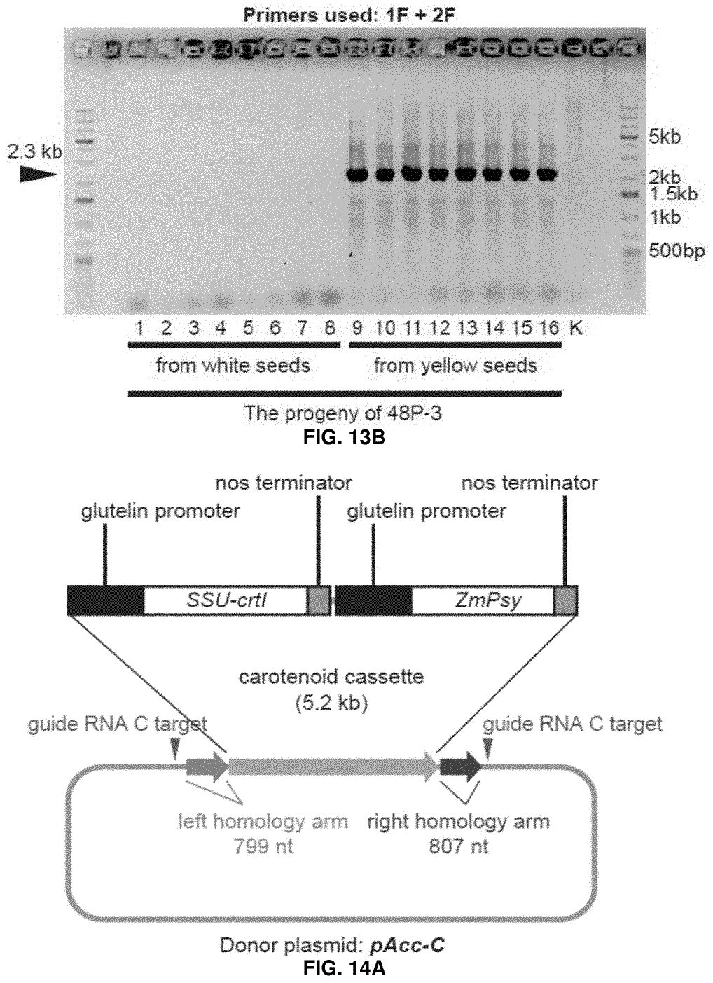

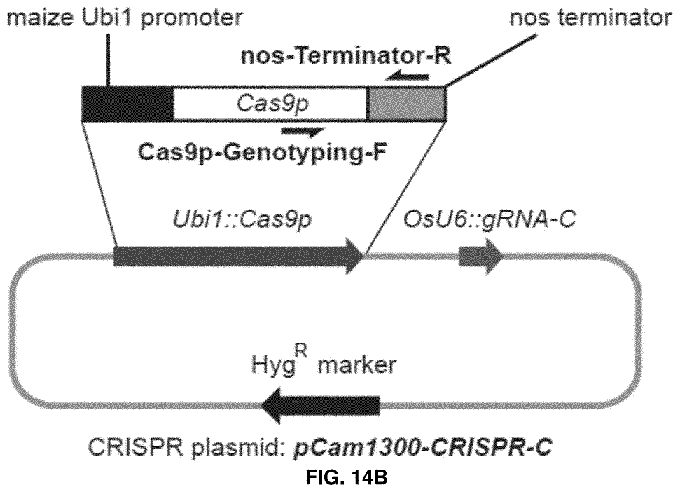

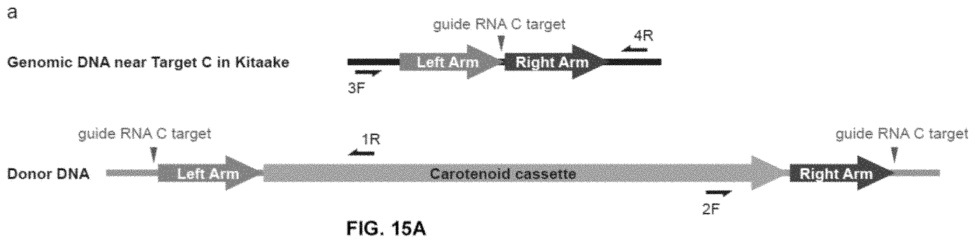

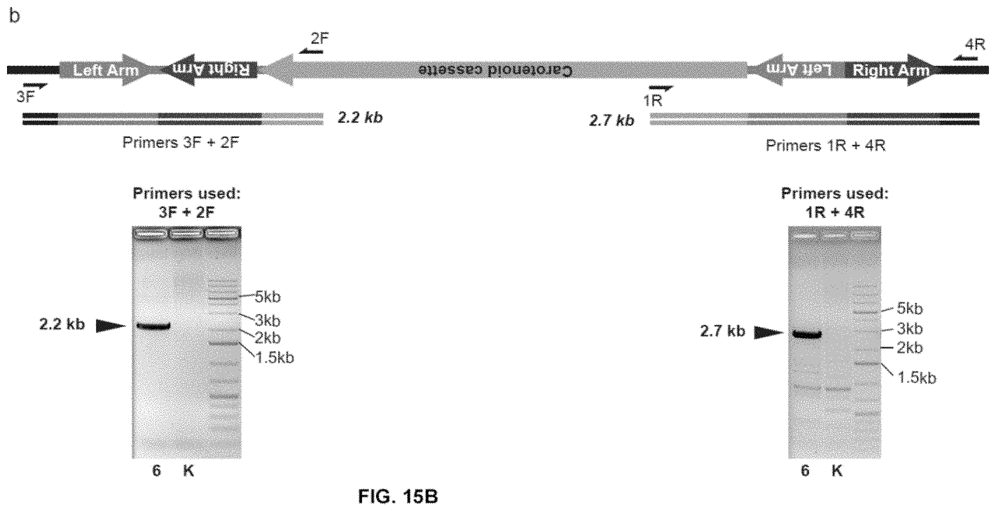

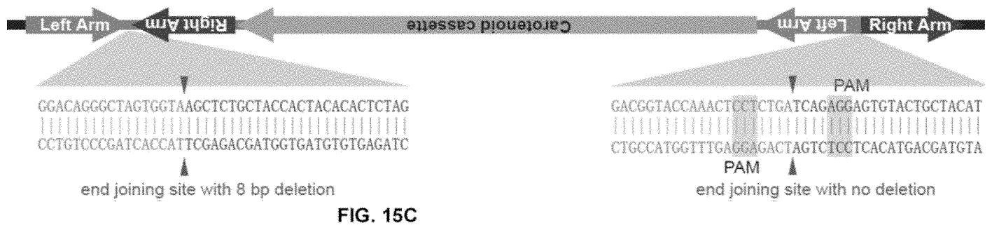

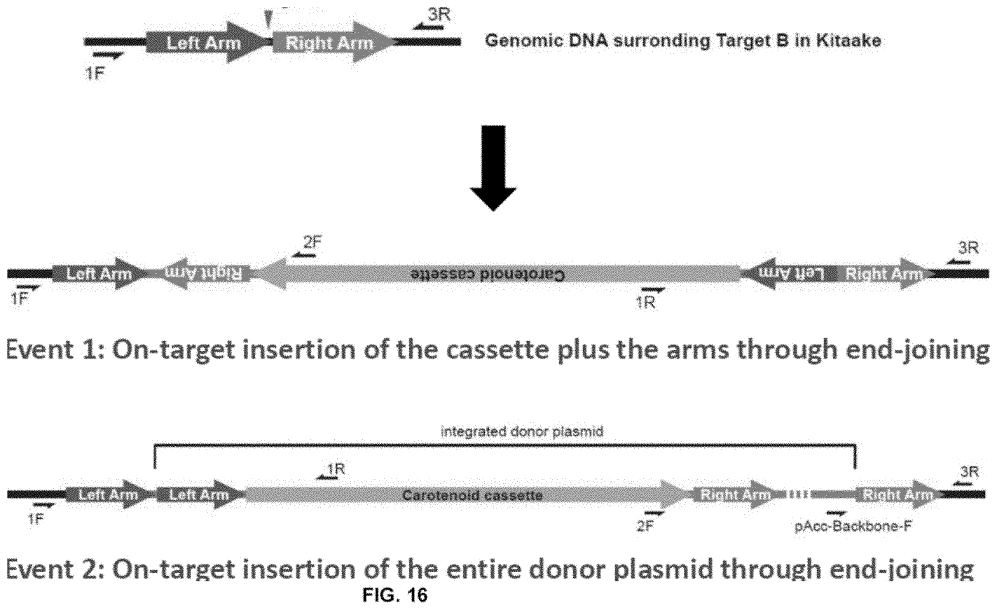

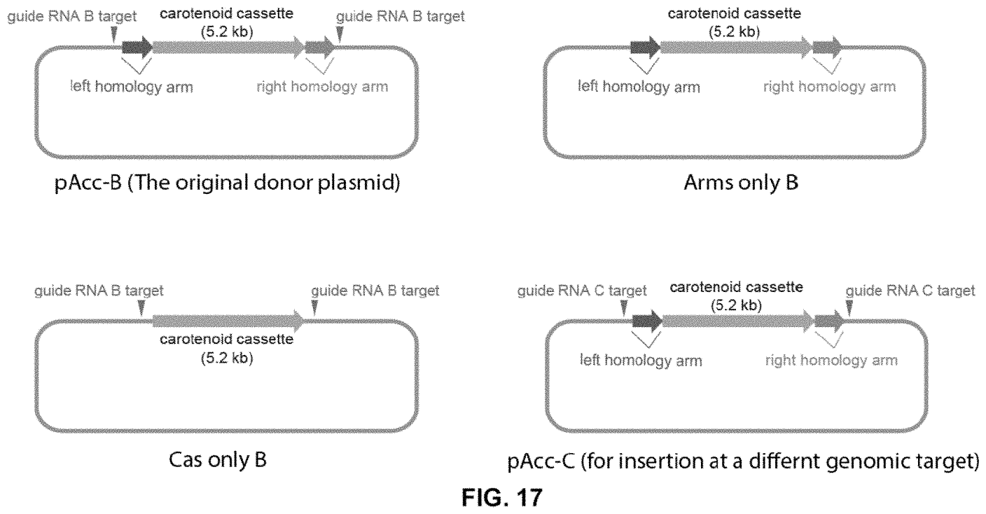

Rice (Oryza sativa) is a staple food crop for more than half of the world's population. To identify GSHs in rice, a mutant screen is conducted by analyzing the morphological records and the whole-genome sequencing data of a fast-neutron rice mutant collection in a model japonica rice variety with short generation time²⁴⁻²⁶. From this screen, five mutants carrying homozygous insertions or translocations (Table 1) are identified, which do not exhibit visible morphological changes compared with the parental genotype. The homozygous mutations in these mutants are verified by PCR using primers flanking the corresponding mutation sites (FIG. 6). Because the mutations do not incur any visible change in morphology, these five intergenic mutation sites (A, B, C, D, and E) are chosen as the candidate GSHs (Table 1).

Using the CRISPR-PLANT guide RNA design platform²⁷, seven specific sites are selected near the five candidate GSHs in the Kitaake rice genome²⁸, and designed guide RNAs (A, B, C, D1, D2, E1, and E2) targeting each of these sites (FIG. 7A). To experimentally determine the ability of Cas9 to induce DSBs at each of the seven targets in vivo, a T7 Endonuclease 1 (T7E1) assay²⁹ is performed in rice protoplasts transiently expressing Cas9 and each of the seven guide RNA candidates. This assay quantifies the frequency of Cas9-induced mutations at each of the seven guide RNAs targets, which reflects the efficiency of cleavage by Cas9 at these targets in vivo. Mutations occurred at targets A, B, and C at relatively high frequencies (FIG. 7B), indicating that targets of guide RNAs A, B, and C are feasible insertion targets for a CRISPR-Cas9-based method.

### Constructing a Maker-Free Carotenoid Cassette for Insertion

Because of the valuable socio-economic impact conferred by the Golden Rice 2 (GR2) cassette, its availability, and the clear phenotype it confers³⁰, this cassette is chosen to be modified and used as the donor DNA to assess the efficiency of marker-free targeted insertion in rice. Rice varieties carrying the Golden Rice 1 (GR1) and the GR2 cassettes accumulate carotenoids in the grain³⁰⁻³². The endosperm of GR1 and GR2 is golden in color^{30,31}, compared with the white endosperm observed in most conventional rice varieties. Consumption of GR1 and GR2 is predicted to have a positive nutritional impact, especially in regions where rice is the major food source and Vitamin A deficiency is prevalent³³. Using the Golden Gate Assembly method³⁴, a carotenoid cassette is generated based on the published sequence of the GR2 cassette³⁰. To reduce the size of the insert, the selectable marker gene and the T-DNA border sequences are not included in the modified cassette. The final 5.2 kb carotenoid cassette (FIG. 1A) consists of the coding sequences of the two carotenoid biosynthetic genes SSU-crtI and ZmPsy³⁰, both driven by the endosperm-specific glutelin promoter³⁰ isolated from the Kitaake rice. SSU-crtI is a functional fusion of the DNA encoding the chloroplast transit peptide from the pea RUBISCO small subunit and the Erwinia uredovora carotenoid desaturase, whereas ZmPsy encodes a maize phytoene synthase. The nopaline synthase (nos) terminator (from Agrobacterium tumefaciens) is used for transcription termination in both genes.

Delivery of the Carotenoid Cassette into Rice at Genomic Targets

The donor plasmid pAcc-B (FIG. 1A) is assembled, which contains the 5.2 kb carotenoid cassette. Homology arms, which consists of 794 bp and 816 bp of Kitaake genomic sequence to the left and right of the Cas9 cleavage site at the guide RNA B target (Target B), respectively, are added. The homology arms are included to facilitate the possibility of homology-directed repair (HDR)³⁵, a precise repair mechanism with relatively low frequency. Two guide RNA B target sequences outside each homology arm on the donor plasmid are placed to further enhance the chance of targeted insertion of the carotenoid cassette sequence, because increased HDR efficiency by linearizing the donor template has previously been observed^{36,37}. It is hypothesized that these guide RNA target sites would facilitate the release of the carotenoid cassette from the circular donor plasmid by Cas9, based on previously described reports^{38,39}.

The nucleotide sequence of pACC-B is shown as follows:

(SEQ ID NO: 1)
CCATTCGCCATTCAGGCTGCGCAACTGTTGGGAAGGGCGATCGGTGCGGGCCTCTTCGCTATTA
CGCCAGCTGGCGAAAGGGGGATGTGCTGCAAGGCGATTAAGTTGGGTAACGCCAGGGTTTTCCC
AGTCACGACGTTGTAAAACGACGGCCAGTGAATTGTAATACGACTCACTATAGGGCGAATTGGG
TACAGTGTGTGGCGCGTGTGAATTCTGATGGAGTTTGGTACCCTCGAGACGTGCAATGGAGTGT
AATACTAATAAAACTTATATAAAAAAAAGTGCACTCCACCTTTCAGGGATTAAAAATAAAAAAT
AAAAATAAAACGGAGCCTTCTAGAAAGAAAAAAAAAACAAAAGAAGCCCACTAAGCTCCGAGAG
GGGTAAAGGAAGGAAGCAGAGGCCCACTCAACCACATGTTGCGTGGACCACAACGGCCCAAAGT
CCGAGGCCCATATTAAACTCCCCCACCGCGTGGCGTGCGCCGTCTTCCTCGAATAGGCGCGAGG
TGGAGGAAGGGCCACCGCCTTATTCTCCGCGACGCCTTCCTCCAAAAGTATCCTCTCGCCATCT
CGCCGCCTCCCCGCGCTGCCCCCTCTCTCCCTCTCCCTCCCTTCTCCGCTCACCTCTCCGTCCG
AATCCAATCCAATCCAATTCAACGTAAGCTCCCTCCCCCGTTCGCGCCTCTCTCCAAATCGCAT
AAGTTTGCCGCTTCGATTCGTGTGTCATGTTTTTTTTTCCTTTTTCTGCTTTGGTTTTTGAAAT
GGGGTTTCGAATTACACTCGTGTCCCCGTTGTTCGCGTTGTATGGAAGTGCGGGATTGTAGAAC
ACGGATTAGATGAATCCTGGTACACACGTCACACGAACGGGTGATGTTGTTGCAAGTGTATAAT
CATCACAGCCATAGGTGGACTGGACTGCGATTGTGTCGTGGATAACTGTATCACCTGAACTCAT
TTGCTTGCGATCTTCATCAACTCAAGTGGTTTATCAAATTGTCTTCCTCCAATTATTGTGGCGC
GTGTGAATTCGGTACCATCCCCAAGTGGTGGCTATCGTTAATCATGGTGTAGGCAACCCAAATA
AAACACCAAAATATGCACAAGGCAGTTTGTTGTATTCTGTAGTACAGACAAAACTAAAAGTAAT
GAAAGAAGATGTGGTGTTAGAAAAGGAAACAATATCATGAGTAATGTGTGAGCATTATGGGACC
ACGAAATAAAAAGAACATTTTGATGAGTCGTGTATCCTCGATGAGCCTCAAAAGTTCTCTCACC
CCGGATAAGAAACCCTTAAGCAATGTGCAAAGTTTGCATTCTCCACTGACATAATGCAAAATAA
GATATCATCGATGACATAGCAACTCATGCATCATATCATGCCTCTCTCAACCTATTCATTCCTA
CTCATCTACATAAGTATCTTCAGCTAAATGTTAGAACATAAACCCATAAGTCACGTTTGATGAG
TATTAGGCGTGACACATGACAAATCACAGACTCAAGCAAGATAAAGCAAAATGATGTGTACATA
AAACTCCAGAGCTATATGTCATATTGCAAAAAGAGGAGAGCTTATAAGACAAGGCATGACTCAC
AAAAATTCATTTGCCTTTCGTGTCAAAAAGAGGAGGGCTTTACATTATCCATGTCATATTGCAA
AAGAAAGAGAGAAAGAACAACACAATGCTGCGTCAATTATACATATCTGTATGTCCATCATTAT
TCATCCACCTTTCGTGTACCACACTTCATATATCATGAGTCACTTCATGTCTGGACATTAACAA
ACTCTATCTTAACATTTAGATGCAAGAGCCTTTATCTCACTATAAATGCACGATGATTTCTCAT
TGTTTCTCACAAAAAGCATTCAGTTCATTAGTCCTACAACAACCCATGAGAATTCGGCTTCCCA
AATCGCCGCCACCATGGCTTCTATGATATCCTCTTCCGCTGTGACAACAGTCAGCCGTGCCTCT
AGGGGGCAATCCGCCGCAGTGGCTCCATTCGGCGGCCTCAAATCCATGACTGGATTCCCAGTGA
AGAAGGTCAACACTGACATTACTTCCATTACAAGCAATGGTGGAAGAGTAAAGTGCATGAAACC
AACTACGGTAATTGGTGCAGGCTTCGGTGGCCTGGCACTGGCAATTCGTCTACAAGCTGCGGGG
ATCCCCGTCTTACTGCTTGAACAACGTGATAAACCCGGCGGTCGGGCTTATGTCTACGAGGATC
AGGGGTTTACCTTTGATGCAGGCCCGACGGTTATCACCGATCCCAGTGCCATTGAAGAACTGTT
TGCACTGGCAGGAAAACAGTTAAAAGAGTATGTCGAACTGCTGCCGGTTACGCCGTTTTACCGC
CTGTGTTGGGAGTCAGGGAAGGTCTTTAATTACGATAACGATCAAACCCGGCTCGAAGCGCAGA
TTCAGCAGTTTAATCCCCGCGATGTCGAAGGTTATCGTCAGTTTCTGGACTATTCACGCGCGGT
GTTTAAAGAAGGCTATCTGAAGCTCGGTACTGTCCCTTTTTTATCGTTCAGAGACATGCTTCGC
GCCGCACCTCAACTGGCGAAACTGCAGGCATGGAGAAGCGTTTACAGTAAGGTTGCCAGTTACA
TCGAAGATGAACATCTGCGCCAGGCGTTTTCTTTCCACTCGCTGTTGGTGGGCGGCAATCCCTT
CGCCACCTCATCCATTTATACGTTGATACACGCGCTGGAGCGTGAGTGGGGCGTCTGGTTTCCG
CGTGGCGGCACCGGCGCATTAGTTCAGGGGATGATAAAGCTGTTTCAGGATCTGGGTGGCGAAG
TCGTGTTAAACGCCAGAGTCAGCCATATGGAAACGACAGGAAACAAGATTGAAGCCGTGCATTT
AGAGGACGGTCGCAGGTTCCTGACGCAAGCCGTCGCGTCAAATGCAGATGTGGTTCATACCTAT
CGCGACCTGTTAAGCCAGCACCCTGCCGCGGTTAAGCAGTCCAACAAACTGCAGACTAAGCGCA
TGAGTAACTCTCTGTTTGTGCTCTATTTTGGTTTGAATCACCATCATGATCAGCTCGCGCATCA
CACGGTTTGTTTCGGCCCGCGTTACCGCGAGCTGATTGACGAAATTTTTAATCATGATGGCCTC
GCAGAGGACTTCTCACTTTATCTGCACGCGCCCTGTGTCACGGATTCGTCACTGGCGCCTGAAG
GTTGCGGCAGTTACTATGTGTTGGCGCCGGTGCCGCATTTAGGCACCGCGAACCTCGACTGGAC
GGTTGAGGGGCCAAAACTACGCGACCGTATTTTTGCGTACCTTGAGCAGCATTACATGCCTGGC
TTACGGAGTCAGCTGGTCACGCACCGGATGTTTACGCCGTTTGATTTTCGCGACCAGCTTAATG
CCTATCATGGCTCAGCCTTTTCTGTGGAGCCCGTTCTTACCCAGAGCGCCTGGTTTCGGCCGCA
TAACCGCGATAAAACCATTACTAATCTCTACCTGGTCGGCGCAGGCACGCATCCCGGCGCAGGC
ATTCCTGGCGTCATCGGCTCGGCAAAAGCGACAGCAGGTTTGATGCTGGAGGATCTGATTTGAG
GCCATGCAGGCCGATCCCCGATCGTTCAAACATTTGGCAATAAAGTTTCTTAAGATTGAATCCT
GTTGCCGGTCTTGCGATGATTATCATATAATTTCTGTTGAATTACGTTAAGCATGTAATAATTA
ACATGTAATGCATGACGTTATTTATGAGATGGGTTTTTATGATTAGAGTCCCGCAATTATACAT
TTAATACGCGATAGAAAACAAAATATAGCGCGCAAACTAGGATAAATTATCGCGCGCGGTGTCA
TCTATGTTACTAGATCGGGCCTTAATAAGCTTCGATCCGTTAATCATGGTGTAGGCAACCCAAA
TAAAACACCAAAATATGCACAAGGCAGTTTGTTGTATTCTGTAGTACAGACAAAACTAAAAGTA
ATGAAAGAAGATGTGGTGTTAGAAAAGGAAACAATATCATGAGTAATGTGTGAGCATTATGGGA
CCACGAAATAAAAAGAACATTTTGATGAGTCGTGTATCCTCGATGAGCCTCAAAAGTTCTCTCA
CCCCGGATAAGAAACCCTTAAGCAATGTGCAAAGTTTGCATTCTCCACTGACATAATGCAAAAT
AAGATATCATCGATGACATAGCAACTCATGCATCATATCATGCCTCTCTCAACCTATTCATTCC
TACTCATCTACATAAGTATCTTCAGCTAAATGTTAGAACATAAACCCATAAGTCACGTTTGATG
AGTATTAGGCGTGACACATGACAAATCACAGACTCAAGCAAGATAAAGCAAAATGATGTGTACA
TAAAACTCCAGAGCTATATGTCATATTGCAAAAAGAGGAGAGCTTATAAGACAAGGCATGACTC
ACAAAAATTCATTTGCCTTTCGTGTCAAAAAGAGGAGGGCTTTACATTATCCATGTCATATTGC
AAAAGAAAGAGAGAAAGAACAACACAATGCTGCGTCAATTATACATATCTGTATGTCCATCATT
ATTCATCCACCTTTCGTGTACCACACTTCATATATCATGAGTCACTTCATGTCTGGACATTAAC
AAACTCTATCTTAACATTTAGATGCAAGAGCCTTTATCTCACTATAAATGCACGATGATTTCTC
ATTGTTTCTCACAAAAAGCATTCAGTTCATTAGTCCTACAACAACTACACTGAATTCGGCTTCC
CAAATCGCCGCCACCATGGCCATCATACTCGTACGAGCAGCGTCGCCGGGGCTCTCCGCCGCCG
ACAGCATCAGCCACCAGGGGACTCTCCAGTGCTCCACCCTGCTCAAGACGAAGAGGCCGGCGGC
GCGGCGGTGGATGCCCTGCTCGCTCCTTGGCCTCCACCCGTGGGAGGCTGGCCGTCCCTCCCCC
GCCGTCTACTCCAGCCTGCCCGTCAACCCGGCGGGAGAGGCCGTCGTCTCGTCCGAGCAGAAGG
TCTACGACGTCGTGCTCAAGCAGGCCGCATTGCTCAAACGCCAGCTGCGCACGCCGGTCCTCGA
CGCCAGGCCCCAGGACATGGACATGCCACGCAACGGGCTCAAGGAAGCCTACGACCGCTGCGGC
GAGATCTGTGAGGAGTATGCCAAGACGTTTTACCTCGGAACTATGTTGATGACAGAGGAGCGGC
GCCGCGCCATATGGGCCATCTATGTGTGGTGTAGGAGGACAGATGAGCTTGTAGATGGGCCAAA
CGCCAACTACATTACACCAACAGCTTTGGACCGGTGGGAGAAGAGACTTGAGGATCTGTTCACG
GGACGTCCTTACGACATGCTTGATGCCGCTCTCTCTGATACCATCTCAAGGTTCCCCATAGACA
TTCAGCCATTCAGGGACATGATTGAAGGGATGAGGAGTGATCTTAGGAAGACAAGGTATAACAA
CTTCGACGAGCTCTACATGTACTGCTACTATGTTGCTGGAACTGTCGGGTTAATGAGCGTACCT
GTGATGGGCATCGCAACCGAGTCTAAAGCAACAACTGAAAGCGTATACAGTGCTGCCTTGGCTC
TGGGAATTGCGAACCAACTCACGAACATACTCCGGGATGTTGGAGAGGATGCTAGAAGAGGAAG
GATATATTTACCACAAGATGAGCTTGCACAGGCAGGGCTCTCTGATGAGGACATCTTCAAAGGG
GTCGTCACGAACCGGTGGAGAAACTTCATGAAGAGGCAGATCAAGAGGGCCAGGATGTTTTTTG
AGGAGGCAGAGAGAGGGGTAACTGAGCTCTCACAGGCTAGCAGATGGCCAGTATGGGCTTCCCT
GTTGTTGTACAGGCAGATCCTGGATGAGATCGAAGCCAACGACTACAACAACTTCACGAAGAGG
GCGTATGTTGGTAAAGGGAAGAAGTTGCTAGCACTTCCTGTGGCATATGGAAAATCGCTACTGC
TCCCATGTTCATTGAGAAATGGCCAGACCTAGGGCCATGCAGGCCGATCCCCGATCGTTCAAAC
ATTTGGCAATAAAGTTTCTTAAGATTGAATCCTGTTGCCGGTCTTGCGATGATTATCATATAAT
TTCTGTTGAATTACGTTAAGCATGTAATAATTAACATGTAATGCATGACGTTATTTATGAGATG
TGTTTTTATGATTAGAGTCCCGCAATTATACATTTAATACGCGATAGAAAACAAAATATAGCGC
GCAAACTAGGATAAATTATCGCGCGCGGTGTCATCTATGTTACTAGATCGTCCAGACTAGTAAG
GGCGAATTCTAGATGATGGACATTGGAGGAGGTTTTGAGCAATCGCACGACTGCTGTTTGGTTT
GCTCTGGCTTTCTGTTTCGCTGAATCTTCATTTCTGATGAAGGGGGTGATATTAAGTTAAACTG
AATTAATTAGACTCTCTGGTGGGGGAAATGAGGTCAATCAACAGCAGATTCAGGCAACTGGCCC
GCTATTAGTCTTCAGTCTGCTTATCGCTATTAGTCTTCAGTCTGCTTATCCTTTTGCGTGATTG
ACATATCACCAACTGTCAGAAGGGGACACATTACAGATAGCTGTAATAAATGATAGATAAGATG
GAATCGAACAGTTGGAGTTTCACTGTGAGACTGCAGCTGGTTGTCTCTTTTTTCAATGTGTAGT
CTGGAAGAACGTTTCTTTCAACTCCTAATGTGCCCCTTCCAGTTATGTGAGCGTGCGATAAGAA
ATCAAGAAGATAACTGTTACATTAGAACTGTCTCACATTTTTTTTTCTTTTCATTTACACCGAA
TCATGGTGAACATGTTCTTCTGCAACAGTGCATTGGCATGCATTATACTAATCATAACCTAAGT
ATTTATCTGATCACAAGCTTTGTCTAATCAGATGAGTAAAAGATTTGAAATCCTTAGCTGATCC
TCCTTGTAATCTCAAGTGTGATGTTTTCAGTTTCAGATGAACAGAGCAGTGATGTTTATGTTGC
TAACAGTATGTGCAATACTCATTGACAGGTGATGCTGCTGCTAGTAGTATCAGAGCTCTACATT
GAAAGATTGCATCTGATTAGGACGGTTCAGGCCAGGGTATGAGCCAAACCTGGTCTTATCTCTC
GAGTCTAGAGTGTGTGGCGCGTGTGAATTCTGATGGAGTTTACTAGAGCGGCCGCCACCGCGGT
GGAGCTCCAGCTTTTGTTCCCTTTAGTGAGGGTTAATTTCGAGCTTGGCGTAATCATGGTCATA
GCTGTTTCCTGTGTGAAATTGTTATCCGCTCACAATTCCACACAACATACGAGCCGGAAGCATA
AAGTGTAAAGCCTGGGGTGCCTAATGAGTGAGCTAACTCACATTAATTGCGTTGCGCTCACTGC
CCGCTTTCCAGTCGGGAAACCTGTCGTGCCAGCTGCATTAATGAATCGGCCAACGCGCGGGGAG
AGGCGGTTTGCGTATTGGGCGCTCTTCCGCTTCCTCGCTCACTGACTCGCTGCGCTCGGTCGTT
CGGCTGCGGCGAGCGGTATCAGCTCACTCAAAGGCGGTAATACGGTTATCCACAGAATCAGGGG
ATAACGCAGGAAAGAACATGTGAGCAAAAGGCCAGCAAAAGGCCAGGAACCGTAAAAAGGCCGC
GTTGCTGGCGTTTTTCCATAGGCTCCGCCCCCCTGACGAGCATCACAAAAATCGACGCTCAAGT
CAGAGGTGGCGAAACCCGACAGGACTATAAAGATACCAGGCGTTTCCCCCTGGAAGCTCCCTCG
TGCGCTCTCCTGTTCCGACCCTGCCGCTTACCGGATACCTGTCCGCCTTTCTCCCTTCGGGAAG
CGTGGCGCTTTCTCATAGCTCACGCTGTAGGTATCTCAGTTCGGTGTAGGTCGTTCGCTCCAAG
CTGGGCTGTGTGCACGAACCCCCCGTTCAGCCCGACCGCTGCGCCTTATCCGGTAACTATCGTC
TTGAGTCCAACCCGGTAAGACACGACTTATCGCCACTGGCAGCAGCCACTGGTAACAGGATTAG
CAGAGCGAGGTATGTAGGCGGTGCTACAGAGTTCTTGAAGTGGTGGCCTAACTACGGCTACACT
AGAAGGACAGTATTTGGTATCTGCGCTCTGCTGAAGCCAGTTACCTTCGGAAAAAGAGTTGGTA
GCTCTTGATCCGGCAAACAAACCACCGCTGGTAGCGGTGGTTTTTTTGTTTGCAAGCAGCAGAT
TACGCGCAGAAAAAAAGGATCTCAAGAAGATCCTTTGATCTTTTCTACGGGGTCTGACGCTCAG
TGGAACGAAAACTCACGTTAAGGGATTTTGGTCATGAGATTATCAAAAAGGATCTTCACCTAGA
TCCTTTTAAATTAAAAATGAAGTTTTAAATCAATCTAAAGTATATATGAGTAAACTTGGTCTGA
CAGTTACCAATGCTTAATCAGTGAGGCACCTATCTCAGCGATCTGTCTATTTCGTTCATCCATA
GTTGCCTGACTCCCCGTCGTGTAGATAACTACGATACGGGAGGGCTTACCATCTGGCCCCAGTG
CTGCAATGATACCGCGAGACCCACGCTCACCGGCTCCAGATTTATCAGCAATAAACCAGCCAGC
CGGAAGGGCCGAGCGCAGAAGTGGTCCTGCAACTTTATCCGCCTCCATCCAGTCTATTAATTGT
TGCCGGGAAGCTAGAGTAAGTAGTTCGCCAGTTAATAGTTTGCGCAACGTTGTTGCCATTGCTA
CAGGCATCGTGGTGTCACGCTCGTCGTTTGGTATGGCTTCATTCAGCTCCGGTTCCCAACGATC
AAGGCGAGTTACATGATCCCCCATGTTGTGCAAAAAAGCGGTTAGCTCCTTCGGTCCTCCGATC
GTTGTCAGAAGTAAGTTGGCCGCAGTGTTATCACTCATGGTTATGGCAGCACTGCATAATTCTC
TTACTGTCATGCCATCCGTAAGATGCTTTTCTGTGACTGGTGAGTACTCAACCAAGTCATTCTG
AGAATAGTGTATGCGGCGACCGAGTTGCTCTTGCCCGGCGTCAATACGGGATAATACCGCGCCA
CATAGCAGAACTTTAAAAGTGCTCATCATTGGAAAACGTTCTTCGGGGCGAAAACTCTCAAGGA
TCTTACCGCTGTTGAGATCCAGTTCGATGTAACCCACTCGTGCACCCAACTGATCTTCAGCATC
TTTTACTTTCACCAGCGTTTCTGGGTGAGCAAAAACAGGAAGGCAAAATGCCGCAAAAAAGGGA
ATAAGGGCGACACGGAAATGTTGAATACTCATACTCTTCCTTTTTCAATATTATTGAAGCATTT
ATCAGGGTTATTGTCTCATGAGCGGATACATATTTGAATGTATTTAGAAAAATAAACAAATAGG
GGTTCCGCGCACATTTCCCCGAAAAGTGCCACCTGACGCGCCCTGTAGCGGCGCATTAAGCGCG
GCGGGTGTGGTGGTTACGCGCAGCGTGACCGCTACACTTGCCAGCGCCCTAGCGCCCGCTCCTT
TCGCTTTCTTCCCTTCCTTTCTCGCCACGTTCGCCGGCTTTCCCCGTCAAGCTCTAAATCGGGG
GCTCCCTTTAGGGTTCCGATTTAGTGCTTTACGGCACCTCGACCCCAAAAAACTTGATTAGGGT
GATGGTTCACGTAGTGGGCCATCGCCCTGATAGACGGTTTTTCGCCCTTTGACGTTGGAGTCCA
CGTTCTTTAATAGTGGACTCTTGTTCCAAACTGGAACAACACTCAACCCTATCTCGGTCTATTC
TTTTGATTTATAAGGGATTTTGCCGATTTCGGCCTATTGGTTAAAAAATGAGCTGATTTAACAA
AAATTTAACGCGAATTTTAACAAAATATTAACGCTTACAATTT

Next, the CRISPR plasmid pCam1300-CRISPR-B is constructed, which consists of a Cas9p module⁴⁰ with a Poaceae (the family of plant that rice belongs to) codon-optimized Cas9-coding sequence driven by the maize Ubiquitin 1 (Ubi1) promoter, and a guide RNA B module driven by the promoter of the rice small nuclear RNA gene OsU6⁴¹ (FIG. 1B). The Cas9p module also includes the nos terminator derived from Agrobacterium. A hygromycin resistance selectable marker gene is present on the backbone of pCam1300-CRISPR-B, which allows for subsequent selection of rice transformants carrying the Cas9-gRNA module using the herbicide hygromycin.

Equal mass of the donor plasmid pAcc-B and the CRISPR plasmid pCam1300-CRISPR-B are mixed and delivered by particle bombardment (FIG. 1C). One hundred Kitaake rice embryogenic calli are bombarded, and applied hygromycin to select for calli transformed with pCam1300-CRISPR-B. 55 hygromycin-resistant plants (T0 generation) are regenerated.

### Insertion of the Carotenoid Cassette Occurred at Target B

The 55 T0 individuals are genotyped by PCR using primers 1F and 1R to check whether carotenoid cassette is inserted at Target B through HDR (FIG. 2A, FIG. 8A). A PCR band for T0 plant #1 at 2.6 kb is observed, which exceeds the size of the predicted band by 0.8 kb, roughly the size of the left homology arm. It is hypothesized that the left junction of this insertion may have occurred through non-homologous end joining (NHEJ), an alternative pathway to repair DSB with a higher frequency compared with HDR³⁵. To test this, additional PCR reactions on T0 plant #1 are performed using primer pairs 1F+1R and 2F+3R (FIGS. 8B and 8C). A 2.6 kb fragment and a 5.2 kb fragment are amplified, respectively. This result suggests that the entire pAcc-B donor plasmid is integrated at Target B in T0 plant #1. Amplicons spanning both junctions of the insert are sequenced to confirm the insertion of the donor plasmid (FIG. 8D). Because T0 plant #1 is sterile, seeds to further validate the nature of the insertion could not be harvested.

To assess the possibility that a subset of the remaining T0 plants also harbored the insertion of the carotenoid cassette through NHEJ but in the opposite orientation, the 55 T0 plants are genotyped using primers 1F and 2F (FIG. 9A). These primers amplified a 2.3 kb band in seven T0 plants (T0 #11, #16, #17, #24, #28, #48, and #50). Both insertion junctions in these seven plants are confirmed by additional PCR reactions using primer pairs 1F+2F and 1R+3R (FIG. 9B). Based on these results, it is predicted that the donor DNA in between the two guide RNA B targets is inserted at Target B through NHEJ in these seven T0 plants (FIG. 9B). By sequencing these amplicons, it is found that the junctions of the inserts in these seven T0 plants are identical (FIG. 2B). The identity of the junctions suggests that these seven T0 plants are likely clonal derivatives of a single independent insertion, which it is subsequently confirmed (see below).

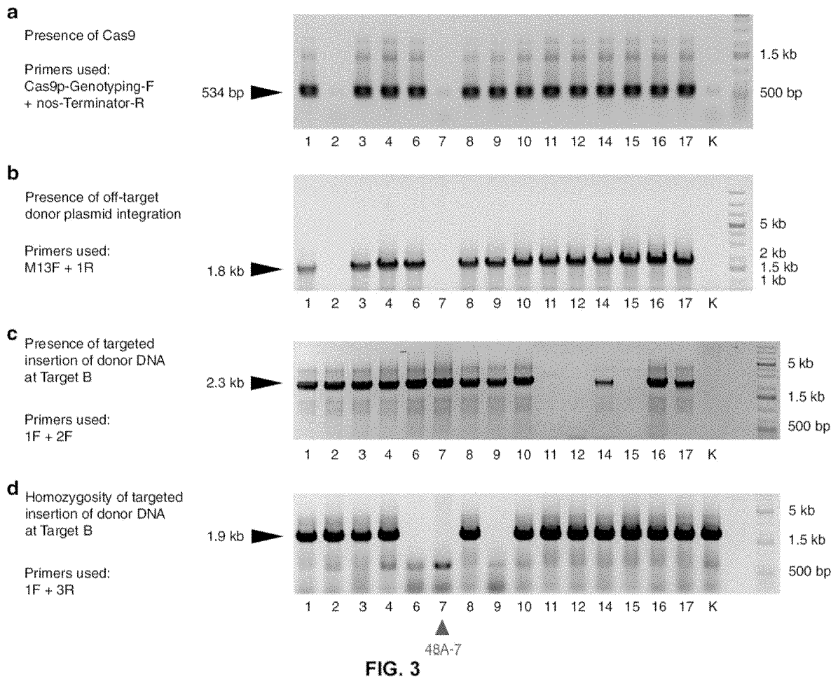

Genetic segregation analysis of the T1 generation is performed to obtain rice plants homozygous for the carotenoid cassette at Target B that lack the Cas9-gRNA module. The progeny of 48A (tiller A from T0 #48) for Cas9 are genotyped using primers “Cas9p-Genotyping-F” and “nos-Terminator-R” located in the Cas9p module (FIG. 1B). In parallel, to detect any potential off-target integration of the donor plasmid during particle bombardment in T0 #48, PCR is performed using the donor backbone-specific primer M13F, and the carotenoid cassette-specific primer 1R (FIG. 1A). In the T1 population, the presence of the Cas9-gRNA module and the backbone of pAcc-B are linked (FIG. 3), suggesting that pAcc-B and pCam1300-CRISPR-B co-integrated in the genome adjacent to each other in plant T0 #48. This result is consistent with the previously reported observation that multiple plasmids frequently co-integrate when delivered through particle bombardment⁴². The same T1 population is next screened for individuals homozygous for the carotenoid cassette at Target B using primers 1F and 3R (FIG. 2A). From these genetic analyses, T1 individual 48A-7 as being homozygous for the inserted carotenoid cassette at Target B and free of the co-integrated CRISPR and donor plasmids are identified (FIG. 3).

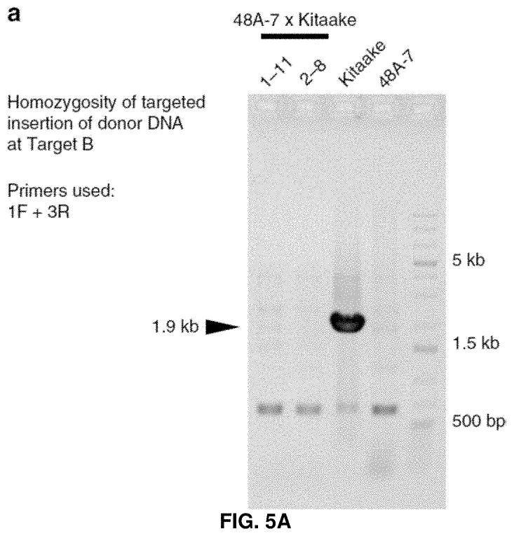

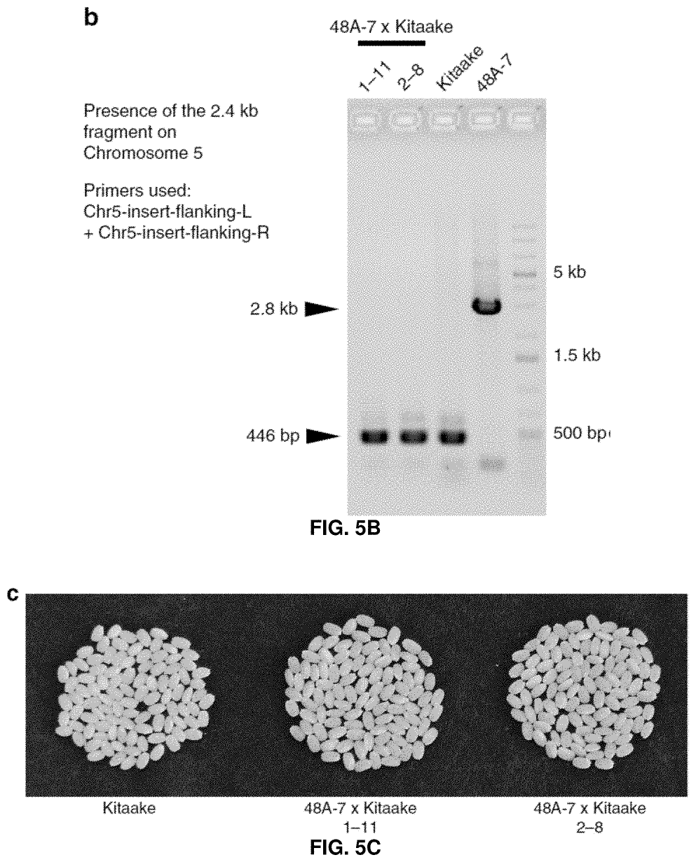

To examine whether the full-length carotenoid cassette is inserted at Target B in 48A-7, PCR is performed using primers 1F and 3R (FIG. 2A) with extended elongation time. A fragment with the expected size of 8.8 kb is amplified (FIG. 5A), indicating that the insert in T0 plant #48 at Target B consists of the full-length carotenoid cassette and both homology arms from the donor plasmid. Consistently, a Southern Blot assay on genomic DNA extracted from 48A-7 supports the presence of a single-copy insertion of the full-length carotenoid cassette and the homology arms at Target B (FIGS. 10B to 10D). The whole-genome sequencing of 48A-7 is also carried out to identify all the sequencing reads (151 bases in length each) that fully or partially match with the sequence of the donor plasmid pAcc-B. These reads are tiled up and the sequence of the insert reconstructed, which is consistent with the sequence of the donor DNA and the Sanger sequencing of the junction ends described in FIG. 2B. In addition, any DNA sequence of the pAcc-B donor plasmid in the rest of the genome of 48A-7 besides Target B is not detected. Together, these results suggest that plant 48A-7 carries a single copy of the full-length carotenoid cassette at Target B.

To assess the occurrence of off-target mutations caused by Cas9 in the process, the whole-genome sequencing result for 48A-7 is further analyzed. Cas-OFFinder⁴³ is used to predict potential Cas9 off-target sites in the KitaakeX genome²⁸ and identified ten candidate sites (Table 3). Sequence analysis indicates that none of the ten predicted off-target sites is mutated in plant 48A-7 (Table 3). This is consistent with the previously reported absence of mutations at predicted Cas9 off-target sites in rice plants edited with CRISPR-Cas9⁴⁴. Together, these results indicate that DNA cleavage by Cas9 is highly specific to Target B in this experiment.

TABLE 3
Absence of mutations at the predicted off-
target sites in 48A-7.

| | | | Number | |
|---|---|---|---|---|
| | | | of | |
| Chromo- | | | Mis- | Mutation |
| some | Position | Sequence | matches | in 48A-7 |
| Chr1 | 42355133 | GTGGCGCGTGTG | 0 | / |
| | | AATTCTGATGG | | |
| Chr8 | 8900142 | GTGGgGCGTGTG | 3 | None |
| | | AAgTgTGAGGG | | |
| Chr1 | 25553130 | GTacCaCGTcTG | 4 | None |
| | | AATTCTGATGG | | |
| Chr2 | 34852771 | GTGGCGttTcTG | 4 | None |
| | | AAcTCTGATGG | | |
| Chr3 | 5583380 | GTGGCGtGcGgG | 4 | None |
| | | AtTTCTGACGG | | |
| Chr3 | 17714129 | GTGGCcCaTaTG | 4 | None |
| | | AgTTCTGAAGG | | |
| Chr3 | 35701456 | GTttCcCtTGTG | 4 | None |
| | | AATTCTGATGG | | |
| Chr5 | 12822698 | aTGGCcCaTGTG | 4 | None |
| | | AgTTCTGAAGG | | |
| Chr6 | 28102882 | GcGGCGCGgGTG | 4 | None |
| | | AAagCTGAGGG | | |
| Chr9 | 11295034 | GaGGCGCGTGTG | 4 | None |
| | | gATcCTGcCGG | | |
| Chr10 | 21956160 | GTGatGCGTGTG | 4 | None |
| | | AATcgTGATGG | | |

Note:
Positions and sequences of the intended insertion target (Target B) and the ten predicted Cas9 off-target sites in the KitaakeX genome.
Nucleotides different from the intended target are indicated by lowercase letters and are colored in red.
The putative protospacer adjacent motif (PAM) sequences are highlighted in yellow.
The sequences above identified as SEQ ID NO: 2-12, respectively.

### Rice Plant 48A-7 Accumulates β-Carotene in the Seed

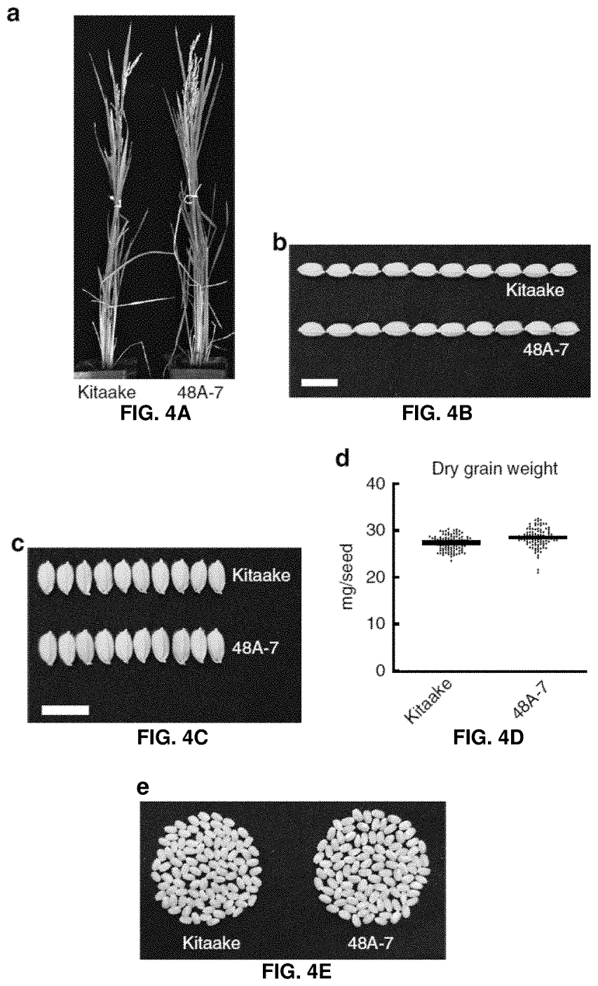

Plant 48A-7 resembles the control plant Kitaake in plant stature and grain dimensions (FIGS. 4A to 4D). The dehusked seeds derived from 48A-7 are golden in color, indicating the accumulation of carotenoids in the endosperm (FIG. 4E). Because the major carotenoid in the endosperm of GR2 is β-carotene³⁰, the β-carotene content is quantified in the endosperm from 48A-7 using high-performance liquid chromatography (HPLC) (FIGS. 11A and 11B). In the dehusked, polished seeds from 48A-7, the β-carotene content is 7.90±0.19 μg g⁻¹ dry weight (Table 4), while no significant amount of β-carotene is detected in the dehusked, polished Kitaake seeds. The observed β-carotene content in 48A-7 is slightly lower than that of the GR2 transformation event GR2E in japonica rice variety Kaybonnet under greenhouse conditions (9.22 μg g⁻¹ dry weight)³⁰, and comparable to the higher end of the range of β-carotene content measured in field-grown indica rice variety PSB Rc82 (1.96-7.31 μg g⁻¹ dry weight)³². The difference in the β-carotene content observed in these studies may be due to the differences in growth conditions, genomic positional effects, and/or post-harvest decay of the carotenoids⁴⁵, the rate of which varies among cultivars⁴⁶. The difference in endogenous carotenoid metabolic components among cultivars may have also contributed to the difference in the level of β-carotene accumulating in the endosperm⁴⁷. Overall, rice plant 48A-7 accumulates a high level of β-carotene in the endosperm.

TABLE 4
Raw data of the β-carotene quantification in rice line 48A-7.

| | | Peak area | | | | |
|---|---|---|---|---|---|---|
| | Area of the β- | conversion to β- | | | | |
| | carotene peak on | carotene quantity | Grain | μg β- | | Standard |
| Rice | HPLC | (μg) based on a | weight | carotene/ | Average | deviation |
| sample | chromatogram | standard curve | (g) | g flour | (μg g⁻¹) | (μg g⁻¹) |
| 48A-7-1 | 662.6 | 0.766 | 0.0989 | 7.74 | 7.90 | 0.19 |
| 48A-7-2 | 696.5 | 0.815 | 0.1038 | 7.85 | | |
| 48A-7-3 | 700.1 | 0.838 | 0.1033 | 8.12 | | |

Note:
Rice seeds harvested from plant 48A-7 are split into three portions, polished, ground to rice flour, and measured separately as technical replicates.

### Obtaining Homozygous Marker-Free Carotenoid-Enriched Rice

Analysis of the whole-genome sequence of 48A-7 revealed a fragment of the CRISPR plasmid at an intergenic region on Chromosome 5, the insertion of which is likely caused by the particle bombardment process⁴⁸. Multiple T0 and T1 plants are genotyped for this insert by PCR using primers flanking the insertion site. A homozygous 2.4 kb insert is detected in 48A-7 (FIG. 12). One copy of this insert is also detected in T0 plants #11, #16, #17, #24, #28, #48, and #50 (FIG. 12). This result indicates that these seven T0 plants are most likely derived from a single transformation event, which carries the 2.4 kb fragment resulting from the particle bombardment process. The 2.4 kb insert is absent from T0 plant #1, which suggests that T0 plant #1 resulted from an independent transformation event (FIG. 12). To remove the 2.4 kb insert from the homozygous carotenoid-enriched rice, the carotenoid-enriched rice line 48A-7 (maternal) with Kitaake (paternal) are backcrossed. The resulting F1 plants are self-pollinated to generate a segregating F2 population. In the F2 generation, two rice plants, 1-11 and 2-8 are identified, as being homozygous for the carotenoid cassette at Target B and free of the 2.4 kb insert (FIGS. 10A and 10B). Seeds harvested from both plants are golden in color (FIG. 10C), indicating that both plants accumulate β-carotene in the endosperm. These marker-free carotenoid-enriched rice plants carry homozygous insertion of the carotenoid cassette at the intended genomic target.

### The β-Carotene Observed is a Consequence of the Carotenoid Cassette at Target B

To confirm that the observed accumulation of β-carotene in the seeds is a consequence of the carotenoid cassette inserted at Target B, a genetic co-segregation analysis is performed. Seeds from 48P-3, a sibling of 48A-7 hemizygous for the insertion at Target B (FIG. 13A) are harvested. A randomly selected tiller from 48P-3 yielded 13 white seeds and 38 golden seeds, which fits the Mendelian ratio of 1:3 for single-site genetic segregation. 8 of the white seeds and 8 of the golden seeds are randomly germinated and genotyped for the presence of the carotenoid cassette at Target B by PCR. The golden seed color co-segregated with the presence of the carotenoid cassette at Target B (FIG. 13B). This indicates that the β-carotene in the seeds from 48A-7 results from the targeted insertion of the carotenoid cassette at Target B.

### Reproducing the Targeted Insertion at a Different Target

To test whether the method of targeted insertion described above can be applied to other chromosomal locations, and to assess the frequency of insertion of the donor DNA, an additional round of co-bombardment experiments is performed at a different target site, Target C. In this experiment, each callus is cultivated separately to prevent clonal propagation. A CRISPR plasmid pCam1300-CRISPR-C and a donor plasmid pAcc-C are generated (FIGS. 14A and 14B) and delivered them to rice calli as described in FIG. 1c. 16 independent T0 events transformed with the CRISPR plasmid regenerated and one event, T0 plant #6, was found to carry the insertion of the carotenoid cassette at Target C (based on PCR genotyping and Sanger sequencing of the PCR products (FIGS. 15A to 15C)). The insertion occurred through non-homologous end joining, similar to the insertion at Target B observed for T0 plant #48 (FIG. 2B). The overall insertion frequency is 1/16 (6.25%), which represents the number of plants with the on-target insertion of the cassette divided by the total number of transgenic T0 plants carrying the Cas9-gRNA module.

### DISCUSSION

Conventional plant genetic engineering methods rely on the insertion of genes encoding desirable agronomic traits at random positions in the genome. This approach can sometimes lead to decreased yields. For example, within GR2-R, one of the independently transformed Golden Rice 2 events, the GR2 cassette is inserted in the first exon of OsAux1, which encodes an auxin transporter essential for plant growth⁴⁹. Homozygous disruption of OsAux1 by the GR2 cassette in GR2-R causes severe developmental defects and a heavy penalty in yield⁴⁹. Incidences like this may potentially be reduced with prior knowledge of GSHs within a given genome, and a reliable tool to insert desired genes at these sites, without disrupting agronomically important genes. In this study, it is demonstrate the feasibility of this strategy by performing targeted insertion of a carotenoid cassette (consisting of two transcription units) at two separate GSHs in rice. This approach could potentially be applied to any crop species with an established transformation protocol, thus offering a promising tool for plant research and for the genetic improvement of crop plants.

A GSH should accommodate transgene without incurring any undesired trait in the resulting transgenic plant. The selection of GSHs in rice is based on the absence of effects on morphology incurred by mutations at these sites in a rice mutant collection^{25,26}. One limitation of this approach is that these selection criteria do not consider the expression level of the transgenes inserted at these sites. To identify GSHs that express transgenes at desired levels, a population of independent transgenic events, each carrying a reporter gene (such as GFP) at a distinct insertion site, can be generated and screened. The homozygous transgenic lines derived from such a study can then be assessed for the expression of the reporter gene as well as agronomic traits. Such a screen would also advance the knowledge of the effects of a particular genomic position on gene expression.

For optimal genetic improvement, it is often desirable to combine multiple transgenes located at different loci to achieve heritable stacked traits in a specific cultivar⁵⁰. Although the advent of marker-assisted selection has improved the accuracy and efficiency of the breeding process, the stacking of transgenes by conventional breeding remains challenging because traits at different loci segregate independently in the progeny⁵¹. In contrast, with targeted gene insertion as reported in this study, genes encoding multiple traits genes can be stacked a single genetic locus, which would simplify the downstream breeding efforts.

It would also be informative to determine whether specific guide RNAs can facilitate large DNA insertions during the DNA-repair process as has been demonstrated for human primary T cells⁵².

### Methods

### Plant Transformation and Growth Conditions

Kitaake, a photoperiod-insensitive cultivar of japonica rice (Oryza sativa sp. japonica) with a short generation time, is used in all experiments²⁴. For germination, seeds are dehusked and sterilized by incubation in 30% bleach for 15 minutes with shaking. The residual bleach is washed away with an equal volume of sterilized water for three times and the seeds are germinated on Murashige and Skoog (MS) media containing 1% sucrose and 0.3% Phytagel (Caisson Labs, Smithfield, Utah) (pH 5.7) in a growth chamber with the temperature set to 28° C. and a 13 h light/11 h dark regime. One-week-old rice seedlings are transferred to an 80/20 (sand/peat) soil mixture in an environmentally-controlled greenhouse with the temperature set to −28-30° C. and humidity to 75-85% with a 14 h light/10 h dark regime for continued growth until mature. Panicles are harvested and dried at 60° C. for 7 days for long-term storage.

For particle bombardment, Kitaake seeds are sterilized and germinated on the MSD medium (MS with 3% sucrose, 2 mg L⁻¹ 2,4-D and 1.2% Agar, pH 5.7) under 28° C. in the dark for 7 days for initial calli induction. Emerging scutella are detached from the seedlings and transferred to fresh MSD medium for continued induction of calli for one month with medium replacement every 10 days. On the day of the bombardment, calli are transferred to the osmotic medium (MS with 3% sucrose, 4.5% mannitol, 4.5% sorbitol, 5 mg L⁻¹ 2,4-D and 0.35% Phytagel (Caisson Labs, Smithfield, Utah)) for osmotic treatment for 4 hours. The bombardment is performed using the PDS-1000/He particle delivery system (Biorad, Hercules, Calif.) according to the user's manual. Donor and CRISPR plasmids are pre-mixed in a 1:1 mass ratio before coated onto the gold particles. Each plate of calli is bombarded twice with 1.0 μm gold particles coated with the plasmids at 900 to 1100 psi with a 6 cm flying distance. After bombardment, the calli are kept on osmotic medium in the dark at 28° C. overnight and then transferred to the MSD medium for recovery at 28° C. for 5 days. Selection and regeneration are performed at 28° C. with a 13 h light/11 h dark regime. For selection, calli are cultured on the MSDH80 medium (MSD with 80 mg L⁻¹ Hygromycin B (A.G. Scientific, San Diego, Calif.)) for 5 weeks. Over this period, calli are moved to fresh MSDH80 medium every 10 days. Calli are regenerated on the MSRH40 medium (MS with 3% sucrose, 0.5 mg L⁻¹ NAA, 3 mg L⁻¹ BAP, 40 mg L⁻¹ Hygromycin B and 1.2% Agar, pH 5.7) for 6 weeks. The regenerated T0 seedlings are transferred to MS medium for rooting for 2 weeks before they are moved to the greenhouse.

### Plasmid Construction

A modular CRISPR-Cas9 toolbox system⁵³ is used to construct the CRISPR plasmids. Briefly, guide RNAs are designed using the CRISPR-PLANT platform²⁷. For each guide RNA designed, a pair of synthesized oligonucleotides, named Target-A/B/C/D1/D2/E1/E2-gRNA F and R, are annealed to form a dimer with overhangs at both ends. Each dimer is ligated with the BsmBI-digested plasmid pYPQ141c (Addgene) to generate an entry clone with the full-length guide RNA. Three-way recombination among pYPQ167 (Addgene, with the Cas9p coding sequence), pYPQ141c (Addgene, with the guide RNA module) and the destination vector backbone is performed using LR Clonase II (Invitrogen, Carlsbad, Calif.) at room temperature for 16 hours. For the protoplast T7E1 assay, the transient expression vector pAHC17⁵⁴ is used as the destination vector. For calli transformation, pCambia1300 (Cambia, Canberra, Australia) is used as the destination vector. Both destination vectors contain the constitutive maize Ubi1⁵⁴ promoter to drive Cas9 in the final constructs. The pAcc starter vector is created by digesting pBluescript II SK (−) (Addgene) with KpnI and XbaI and ligating the digestion product with an oligodimer formed between primers pAcc-Engineer-F and pAcc-Engineer-R. To construct the donor plasmid, the carotenoid cassette is assembled by ligating four PCR fragments and the pAcc plasmid backbone. The names of the eight PCR primers used to generate the four fragments are Cassette-AF/AR/BF/BR/CF/CR/DF/DR. An oligodimer of guide RNA target nucleotide sequence is ligated into the donor plasmid on each side of the carotenoid cassette using BbsI and BsmBI, respectively. The names of these primers are Target-B/C-PAM-F/R. The left and right homology arms are PCR-amplified and ligated into the donor plasmid by KpnI and XbaI, respectively. The names of the primers used to amplify the homology arms are B/C-Left/Right Arm-F/R. All plasmids are validated by Sanger sequencing (Quintara Biosciences, San Francisco, Calif.) and by checking the electrophoresis patterns after restriction digestion with the Fast Digest enzymes (Thermo Fisher, Waltham, Mass.). The sequences of all primers can be found in Table 2.

TABLE 2
Primer used in this example.

| Name | Sequence |
|---|---|
| Target-A-gRNA-F | GTGTGCAAGGCACTCAACTACGTG |
| | (SEQ ID NO: 13) |
| Target-A-gRNA-R | AAACCACGTAGTTGAGTGCCTTGC |
| | (SEQ ID NO: 14) |
| Target-B-gRNA-F | GTGTGTGGCGCGTGTGAATTCTGA |
| | (SEQ ID NO: 15) |
| Target-B-gRNA-R | AAACTCAGAATTCACACGCGCCAC |
| | (SEQ ID NO: 16) |
| Target-C-gRNA-F | GTGTGTAGTGGTAGCAGAGCTCAG |
| | (SEQ ID NO: 17) |
| Target-C-gRNA-R | AAACCTGAGCTCTGCTACCACTAC |
| | (SEQ ID NO: 18) |
| Target-D1-gRNA-F | GTGTGTGGTTTTCGAACATACGGT |
| | (SEQ ID NO: 19) |
| Target-D1-gRNA-R | AAACACCGTATGTTCGAAAACCAC |
| | (SEQ ID NO: 20) |
| Target-D2-gRNA-F | GTGTGGTGGTTTACAAACATACGA |
| | (SEQ ID NO: 21) |
| Target-D2-gRNA-R | AAACTCGTATGTTTGTAAACCACC |
| | (SEQ ID NO: 22) |
| Target-E1-gRNA-F | GTGTGCGGGGAGAAGAACGAGACT |
| | (SEQ ID NO: 23) |
| Target-E1-gRNA-R | AAACAGTCTCGTTCTTCTCCCCGC |
| | (SEQ ID NO: 24) |
| Target-E2-gRNA-F | GTGTGGAAGACGCCTTCGACAGGT |
| | (SEQ ID NO: 25) |
| Target-E2-gRNA-R | AAACACCTGTCGAAGGCGTCTTCC |
| | (SEQ ID NO: 26) |
| Target-A-PCR-F | GCCTGACAGTGCGTGGTC |
| | (SEQ ID NO: 27) |
| Target-A-PCR-R | GCCTCATCGCTCCTCGTGAT |
| | (SEQ ID NO: 28) |
| Target-B-PCR-F | GACAGTTGGTGATATGTCAATCACGC |
| | (SEQ ID NO: 29) |
| Target-B-PCR-R | TTTGCCGCTTCGATTCGTGT |
| | (SEQ ID NO: 30) |
| Target-C-PCR-F | GAATAGCAGAGTCCACGAGACGA |
| | (SEQ ID NO: 31) |
| Target-C-PCR-R | TTTAGAGTACGTGGGCACGTCG |
| | (SEQ ID NO: 32) |
| Target-D-PCR-F | TTCGGATGTGAACAATACACTGCTAT |
| | (SEQ ID NO: 33) |
| Target-D-PCR-R | ACATTAGAATCCATTTCCATAATTAAGGG |
| | (SEQ ID NO: 34) |
| Target-E-PCR-F | GGCGACGGCAAACCCGATG |
| | (SEQ ID NO: 35) |
| Target-E-PCR-R | GGCCACGCCTCCTGCACTA |
| | (SEQ ID NO: 36) |
| Cassette-AF | CGGGGTACCGGTCTCGCTATCGTTAATCA |
| | TGGTGTAGGCAA (SEQ ID NO: 37) |
| Cassette-AR | GCTCTAGAGGTCTCACATGGGTTGTTGTA |
| | GGACTAATGAACTG (SEQ ID NO: 38) |
| Cassette-CF | CGGGGTACCGGTCTCAGATCCGTTAATCA |
| | TGGTGTAGGCAA (SEQ ID NO: 39) |
| Cassette-CR | GCTCTAGAGGTCTCAGTGTAGTTGTTGTA |
| | GGACTAATGAACTG (SEQ ID NO: 40) |
| Cassette-BF | CGGGGTACCGGTCTCACATGAGAATTCGG |
| | CTTCCCAAATC (SEQ ID NO: 41) |
| Cassette-BR | GCTCTAGAGGTCTCAGATCGAAGCTTATT |
| | AAGGCCCGATC (SEQ ID NO: 42) |
| Cassette-DF | CGGGGTACCGGTCTCTACACTGAATTCGG |
| | CTTCCCAAAT (SEQ ID NO: 43) |
| Cassette-DR | GCTCTAGAGGTCTCTCTGGACGATCTAGT |
| | AACATAGATGACACC (SEQ ID NO: |
| | 44) |
| Target-B-PAM-F | GTGTGTGGCGCGTGTGAATTCTGATGGA |
| | (SEQ ID NO: 45) |
| Target-B-PAM-R | AAACTCCATCAGAATTCACACGCGCCAC |
| | (SEQ ID NO: 46) |
| Target-C-PAM-F | GTGTGTAGTGGTAGCAGAGCTCAGAGGA |
| | (SEQ ID NO: 47) |
| Target-C-PAM-R | AAACTCCTCTGAGCTCTGCTACCACTAC |
| | (SEQ ID NO: 48) |
| B-Left-Arm-F | CGGGGTACCCTCGAGACGTGCAATGGAG |
| | TGTAATAC (SEQ ID NO: 49) |
| B-Left-Arm-R | CGGGGTACCGAATTCACACGCGCCACAA |
| | T (SEQ ID NO: 50) |
| B-Right-Arm-F | TGCTCTAGATGATGGACATTGGAGGAGG |
| | TT (SEQ ID NO: 51) |
| B-Right-Arm-R | TGCTCTAGACTCGAGAGATAAGACCAGG |
| | TTTGGC (SEQ ID NO: 52) |
| C-Left-Arm-F | CGGGGTACCGTCAGGATTGGTAGGAATT |
| | AGC (SEQ ID NO: 53) |
| C-Left-Arm-R | CGGGGTACCAGCTCTGCTACCACTAGCC |
| | (SEQ ID NO: 54) |
| C-Right-Arm-F | TGCTCTAGAGAGGAGTGTACTGCTACAT |
| | GATG (SEQ ID NO: 55) |
| C-Right-Arm-R | TGCTCTAGAAGAGGTAGGACGTTACAGG |
| | TGT (SEQ ID NO: 56) |
| 1F | ATTGGCAACGCACTGGATTC |
| | (SEQ ID NO: 57) |
| 2F | GGCAGATCCTGGATGAGATC |
| | (SEQ ID NO: 58) |
| 1R | GGAAGCCGAATTCTCATGG |
| | (SEQ ID NO: 59) |
| 3R | CTCATGGAAGGTGCACAACC |
| | (SEQ ID NO: 60) |
| 3F | AGGACGGAGAAAGTACTGCATAG |
| | (SEQ ID NO: 61) |
| 4R | AAGAACTCCGAGGTTAAAGCG |
| | (SEQ ID NO: 62) |
| M13F | GTAAAACGACGGCCAGT |
| | (SEQ ID NO: 63) |
| Cas9p-Genotyping- | CGAGAACATCATCCACCTCTTCA |
| F | (SEQ ID NO: 64) |
| nos-Terminator-R | ATCTAGTAACATAGATGACACCGCG |
| | (SEQ ID NO: 65) |
| pAcc-Engineer-F | AGTGTGGGTCTTCGAGAAGACCTGT |
| | TTGGTACCCCGCTAGTCTAGAGTGT |
| | GGAGACGATTGCGTCTCTGTTTA |
| | (SEQ ID NO: 66) |
| pAcc-Engineer-R | CTAGTAAACAGAGACGCAATCGTCT |
| | CCACACTCTAGACTAGCGGGGTACC |
| | AAACAGGTCTTCTCGAAGACCCACA |
| | CTGTAC (SEQ ID NO: 67) |
| Chr5-insert- | AATAACAGAGAGGCTGAGAGTC |
| flanking-L | (SEQ ID NO: 68) |
| Chr5-insert- | GGAGAAGCGTGGGAATAAGAA |
| flanking-R | (SEQ ID NO: 69) |

### Plant DNA Isolation and Genotyping

Genomic DNA is isolated from rice leaf tissue using the cetyltrimethylammonium bromide (CTAB)-chloroform-based method²⁶. For each T0 plant, leaf segments from all tillers are harvested and pooled. For all other plants, a single leaf segment is harvested. PCR genotyping is performed using the DreamTaq (Thermo Fisher, Waltham, Mass.). The GeneRuler 1 kb DNA Ladder (Thermo Fisher, Waltham, Mass.) is used in all DNA electrophoresis experiments. The Sanger sequencing service is provided by Quintara Biosciences (San Francisco, Calif.). The sequences of all primers can be found in Table 2.

### Southern Blotting

5 μg of rice genomic DNA is digested overnight using the restriction enzyme BamHI (New England Biolabs, Ipswich, Mass.) for 16 hours at 37° C. Digested DNA is recovered through ethanol precipitation and split into two equal portions and is run on two duplicate 0.7% agarose gels in parallel. One gel is subject to ethidium bromide staining to determine the completeness of the restriction digestion. The other gel is subject to Southern Blotting with the following procedure. Depurination is performed by rocking the gel in 100 mL of 125 mM HCl for 15 min at room temperature. Denaturation is performed by rocking the gel in 400 mL of the Denaturation Buffer (0.5 M NaOH, 1.5 M NaCl) at room temperature for 1 hour. After denaturation, the gel is neutralized in the Neutralization Buffer (1.5 M NaCl, 0.5 M Tris-HCl, pH 7.5) for 30 minutes with shaking. Overnight blotting of the denatured DNA from the gel onto the Hybond-NX (GE Healthcare, Chicago, Ill.) membrane is performed in 10×SSC Solution (1.5 M NaCl, 150 mM trisodium citrate, pH 7.0). Transferred DNA is crosslinked to the membrane with a UV crosslinker with the energy set to 70000 μJ cm⁻². Pre-hybridization treatment of the membrane is performed in a hybridization tube using the DIG Esay-Hyb Buffer (MilliporeSigma, Burlington, Mass.) at 42° C. with rotation for 30 minutes. The hybridization probe is labeled with DIG using the DIG DNA Labeling Kit (MilliporeSigma, Burlington, Mass.), and denatured by incubating at 95° C. for 5 minutes and immediately chilling on ice. The denatured probe is added to the above-mentioned hybridization tube with the membrane and the DIG Esay-Hyb Buffer. Hybridization is performed at 42° C. for 24 hours with rotation. The membrane is washed in 2×SSC (0.3 M NaCl, 30 mM trisodium citrate, pH 7.0) with 0.1% SDS twice at room temperature, 5 minutes each. The membrane is then washed in 0.1×SSC (0.3 M NaCl, 30 mM trisodium citrate, pH 7.0) with 0.1% SDS twice at 65° C., 15 minutes each. After washing, the blot is processed using the DIG Luminescent Detection Kit (MilliporeSigma, Burlington, Mass.). The Chemiluminescent signal is detected with the ChemiDoc XRS (Biorad, Hercules, Calif.).

### Rice Protoplast Transformation and the T7E1 Assay

Rice protoplasts are prepared from 10-day-old Kitaake seedlings^{29,55}. Rice leaf tissue is cut into 0.5 mm-long pieces using a razor blade. Leaf pieces are incubated in the Enzyme Solution (20 mM MES pH 5.7, 10 mM KCl, 0.6 M mannitol, 1.5% Cellulase Onozuka R-10 (RPI Research Products, Mount Prospect, Ill.), 0.75% Macerozyme R-10 (RPI Research Products, Mount Prospect, Ill.), 0.1% BSA) in the dark at 25° C. in a sterile Erlenmeyer flask with gentle shaking for 6 hours. An equal volume of the W5 solution (154 mM NaCl, 125 mM CaCl2, 5 mM KCl, 2 mM MES pH 5.7) is added to the digest, and the released protoplasts are collected by filtering the digest through a 40-μm nylon mesh. The cells are spun down at 250 g for 3 minutes at room temperature, and the pellet is washed three times with the W5 solution. The protoplasts are re-suspended in the Mmg Solution (0.4 M Mannitol, 15 mM MgCl2, 4 mM MES pH 5.7) to reach 2.5×10⁶ cells mL⁻¹. To initiate transformation, 200 μL of the polyethylene glycol (PEG) Solution (40% PEG 4000, 0.2 M Mannitol, 100 mM CaCl2) is mixed with 200 μL cell resuspension in an Eppendorf tube in the presence of the CRISPR plasmid DNA at a final concentration of 10 ng μL⁻¹. The transformation process continued in the dark at 25° C. for 20 minutes. The transformation is terminated by adding 800 μL of the W5 Solution to the cell resuspension and mixing by inverting the tube. Cells are spun down at 250 g for 3 minutes at room temperature and the supernatant is discarded. Cells from each tube are resuspended in 2 mL of the WI Solution (0.5 M Mannitol, 20 mM KCl, 4 mM MES pH 5.7, 25 μg mL⁻¹ carbenicillin) and kept in the dark at 25° C. for 70 hours. Genome DNA is then extracted from the protoplast cells using the CTAB-chloroform-based method. For the T7E1 assay, genomic DNA fragments spanning various targets are amplified with the Phusion High-Fidelity DNA Polymerase System (Thermo Fisher, Waltham, Mass.) using primers Target-A/B/C/D/E-PCR-F and R. The PCR products are heated to 95° C. and ramped down to 25° C. over 14 minutes evenly to allow heteroduplex formation. T7 endonuclease I (New England Biolabs, Ipswich, Mass.) digestion is performed for 20 minutes at 37° C. The digestion product is separated by electrophoresis on a 2% agarose gel. The intensity of the bands is quantified using Image J (National Institute of Health), which is used to calculate the proportion of digested DNA. The sequences of all primers can be found in Table 2.

### Carotenoid Extraction and HPLC Analysis

Rice seeds are dried at ambient temperature for two weeks after harvest. Dehusked rice grains are polished using sandpaper and ground into flour in liquid nitrogen using mortar and pestle. About 100 mg of rice flour is rehydrated in 200 μL of water prior to carotenoid extraction and 2.5 μg of β-apo-8′-carotenal is added to each sample for estimation of recovery rate. After incubation in the dark at room temperature for 10 min, the rehydrated rice flour is extracted in 1.25 mL methanol with mixing by a vortex and followed by an additional incubation in the dark for 5 minutes. The methanolic extract is centrifuged at 13,000 g for 5 minutes and the supernatant is transferred to a new tube. The pellet is re-extracted with 1.5 mL of diethyl ether twice. The diethyl ether extracts are pooled, combined with the methanolic supernatant, and phase-separated by adding 2 mL of water. The upper phase is saved; the water phase is re-extracted with 1.5 mL of diethyl ether and pooled with the upper phase. The diethyl ether extract is dried under nitrogen gas and the carotenoid residues are resuspended in 320 μL of ethyl acetate. The carotenoid extract (20 μL) is injected on a reverse-phase HPLC and the sample separations are performed⁵⁶. Gradients between two solvents (A) acetonitrile:water:triethylamine (900:99:1, v/v/v) and (B) ethyl acetate are used in HPLC separation, and at a flow rate of 1 mL min-1. The HPLC gradient is 0-5 min, 100-75% A; 5-10 min, 75-30% A; 10-15 min, 30-0% A; 15-16 min, 0-100% A, and 16-17 min, 100% A.

### Analysis of Plasmid Insertions in 48A-7

For whole-genome sequencing, genomic DNA is isolated from 48A-7 for library construction. The sequencing reaction is performed using the HiSeq 2500 sequencing system (Illumina, San Diego, Calif.) at the Joint Genome Institute following the manufacturer's instructions²⁶. A BLAST search against the whole-genome sequencing reads is performed for 48A-7 using the donor DNA sequence consisting of the carotenoid cassette plus the two homology arms as the query. Matching reads are identified and overlaid based on overlapping sequences to confirm the structure of the insert at Target B in 48A-7. To examine whether any plasmid DNA is present in the genome at sites other than Target B, all whole-genome sequencing reads are screened for the ones matching the donor plasmid or the CRISPR plasmid and identified the position of these reads in the KitaakeX genome by BLAST. The carotenoid-enriched rice described in this study is generated in the Kitaake genetic background. KitaakeX, a rice genotype whose genome is avialbale²⁸, carries an XA21 transgene in the Kitaake genetic background. The KitaakeX reference genome²⁸ is used as a close approximation of the Kitaake genome in this study.

### Predicting Potential Cas9 Off-Target Mutation Sites in 48A-7

The KitaakeX genome²⁸ ([webpage for: phytozome.jgi.doe.gov/]) is screened for potential off-target mutation sites using the Cas-OFFinder⁴³ with default parameters. All potential Cas9 off-target sites with four or fewer nucleotide mismatches compared with the true target of guide RNA B are called.

### Analysis of Genomic Variants Between 48A-7 and Kitaake

The paired-end reads for 48A-7 are mapped to the KitaakeX²⁸ rice reference genome using the mapping tool Borrow Wheeler Aligner (BWA version 0.5.9) with default parameters⁵⁷. Genomic variants, including single nucleotide variations (SNVs), deletions, and small insertions are called. To call SNVs and small insertions/deletions (<30 bp), SAMtools mpileup (-E-C 50-DS-m 2-F 0.010638-d 50000) version 0.1.19+ and bcftools (-bcgv-p 0.989362) are used for the merged dataset and filtered using vcfutils.pl (-D 50000-w0-W0-10-20-40-e0) from the SAMtools package⁵⁸. The minimum QUAL score is 100 for SNVs. Pindel version 0.2.4w⁵⁹ is run with default parameters using BreakDancer⁶⁰ results as the input. Small insertions/deletions simultaneously called by SAMtools and Pindel are kept; those called only by SAMtools are filtered at a QUAL score of 100, and those only called by Pindel are further filtered with the following criteria: 1) the variation site had a minimum 10 reads, 2) at least 30% of the reads supported the variation. From the variants between 48A-7 and KitaakeX, the known variants between Kitaake and KitaakeX²⁸ are subtracted. The remaining are the true variants between 48A-7 and its genetic background Kitaake. These genomic variants are compared with the predicted Cas9 off-target sites to evaluate the occurrence of Cas9-induced off-target mutations.

### Data Availability

The whole-genome sequencing data for 48A-7 have been deposited into NCBI's Sequence Read Archive (SRP174336, [webpage for: ncbi.nlm.nih.gov/sra/SRP174336]).

### Example 2

### Targeted Insertion of a Large DNA Fragment in Rice

The strategy taken is shown in FIGS. 1A to 1C. Delivery is through co-bombardment of the CRISPR and the donor plasmids. The insertion can be identified without selection (marker-free DNA insert).

The initial results are shown in FIG. 16. Homozygous line (48A7) from Event 1 is obtained by genetic segregation. See FIG. 4A to 4D. The following are observed: homozygous for the insert at the target, free of the CRISPR genes, no yield penalty, and high beta-carotene content.

To determine whether the homology arms (Arms) and the guide RNA target sites (Cas) on the donor plasmid, the insertion experiments is repeated with alternative donor plasmids. See FIG. 17. The results are shown in Table 5.

TABLE 5
Summary of results.

| Donor used in the | | Cas9-positive T0s | Targeted insertion of |
|---|---|---|---|
| bombardment | Total calli shot | (Transformation %) | the carotenoid cassette |
| Target B Arms only | 275 | 41 (14.9%) | 0 |
| Target B Cas only | 497 | 30 (6.0%) | 0 |
| Target B Cas + arms | 581 | *56+ (9.6%) | 1 (<1.8%) |
| (pAcc-B) | | | |
| Target C Cas + arms | 124 | 16 (12.9%) | 1 (6.25%) |
| (pAcc-C) | | | |

*This number is possibly an underestimation as all potential colonal propagants are counted as a single event. Therefore, the overall efficiency for this treatment is estimanted to be up to 1.8%

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method for inserting a DNA fragment into a target location in a genome of a plant cell, said method comprising: (a) providing a composition comprising an first nucleic acid encoding a cassette comprising one or more genes of interest (GOI) flanked by a left arm 5′ of the cassette and a right arm 3′ of the cassette, and a first target sequence 5′ of the left arm and a second target sequence 3′ of the right arm, and a second nucleic acid encoding a Cas9p, gRNA-B, and a marker; (b) introducing the first nucleic acid and the second nucleic acid into a plant cell, and (c) growing the plant cell on a medium that selects for the marker.

2. The method of claim 1, wherein the plant cell is a plant calli.

3. The method of claim 2, wherein the method further comprises: (d) regenerating a seedling from the plant calli such that the seedling grows a plant.

4. The method of claim 3, wherein the method further comprises: (e) confirming the insertion of the GOI in a targeted location of the genome of the plant regenerating seedlings from the plant calli.

5. The method of claim 1, wherein the GOI is one or more enzymes.

6. The method of claim 5, wherein the one or more enzymes are one or more biosynthetic enzymes.

7. The method of claim 6, wherein the one or more biosynthetic enzymes are from a biosynthetic pathway for producing a compound.

8. The method of claim 7, wherein the compound is a carotenoid.

9. The method of claim 1, wherein the marker is an antibiotic resistance marker.

10. The method of claim 1, wherein the plant cell is a monocot.

11. The method of claim 10, wherein the monocot is a grass.

12. The method of claim 11, wherein the grass is a rice.

13. A composition comprising an isolated first nucleic acid encoding a cassette comprising one or more genes of interest (GOI) flanked by a left arm 5′ of the cassette and a right arm 3′ of the cassette, and a first guide RNA target sequence 5′ of the left arm and a second guide RNA target sequence 3′ of the right arm.

14. The composition of claim 13, the composition further comprises an isolated second nucleic acid encoding a Cas9p, a guide RNA, and a selectable marker.


SEQUENCE LISTING (82 sequences)

SEQ ID NO: 1 - DNA, 9835 bp, Artificial Sequence
  Plasmid pAcc-B which contains carotenoid cassette with a left homology arm flanked by guide RNA B targets.
ccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggtacagtgtgtggcgcgtgtgaattctgatggagtttggtaccctcgagacgtgcaatggagtgtaatactaataaaacttatataaaaaaaagtgcactccacctttcagggattaaaaataaaaaataaaaataaaacggagccttctagaaagaaaaaaaaaacaaaagaagcccactaagctccgagaggggtaaaggaaggaagcagaggcccactcaaccacatgttgcgtggaccacaacggcccaaagtccgaggcccatattaaactcccccaccgcgtggcgtgcgccgtcttcctcgaataggcgcgaggtggaggaagggccaccgccttattctccgcgacgccttcctccaaaagtatcctctcgccatctcgccgcctccccgcgctgccccctctctccctctccctcccttctccgctcacctctccgtccgaatccaatccaatccaattcaacgtaagctccctcccccgttcgcgcctctctccaaatcgcataagtttgccgcttcgattcgtgtgtcatgtttttttttcctttttctgctttggtttttgaaatggggtttcgaattacactcgtgtccccgttgttcgcgttgtatggaagtgcgggattgtagaacacggattagatgaatcctggtacacacgtcacacgaacgggtgatgttgttgcaagtgtataatcatcacagccataggtggactggactgcgattgtgtcgtggataactgtatcacctgaactcatttgcttgcgatcttcatcaactcaagtggtttatcaaattgtcttcctccaattattgtggcgcgtgtgaattcggtaccatccccaagtggtggctatcgttaatcatggtgtaggcaacccaaataaaacaccaaaatatgcacaaggcagtttgttgtattctgtagtacagacaaaactaaaagtaatgaaagaagatgtggtgttagaaaaggaaacaatatcatgagtaatgtgtgagcattatgggaccacgaaataaaaagaacattttgatgagtcgtgtatcctcgatgagcctcaaaagttctctcaccccggataagaaacccttaagcaatgtgcaaagtttgcattctccactgacataatgcaaaataagatatcatcgatgacatagcaactcatgcatcatatcatgcctctctcaacctattcattcctactcatctacataagtatcttcagctaaatgttagaacataaacccataagtcacgtttgatgagtattaggcgtgacacatgacaaatcacagactcaagcaagataaagcaaaatgatgtgtacataaaactccagagctatatgtcatattgcaaaaagaggagagcttataagacaaggcatgactcacaaaaattcatttgcctttcgtgtcaaaaagaggagggctttacattatccatgtcatattgcaaaagaaagagagaaagaacaacacaatgctgcgtcaattatacatatctgtatgtccatcattattcatccacctttcgtgtaccacacttcatatatcatgagtcacttcatgtctggacattaacaaactctatcttaacatttagatgcaagagcctttatctcactataaatgcacgatgatttctcattgtttctcacaaaaagcattcagttcattagtcctacaacaacccatgagaattcggcttcccaaatcgccgccaccatggcttctatgatatcctcttccgctgtgacaacagtcagccgtgcctctagggggcaatccgccgcagtggctccattcggcggcctcaaatccatgactggattcccagtgaagaaggtcaacactgacattacttccattacaagcaatggtggaagagtaaagtgcatgaaaccaactacggtaattggtgcaggcttcggtggcctggcactggcaattcgtctacaagctgcggggatccccgtcttactgcttgaacaacgtgataaacccggcggtcgggcttatgtctacgaggatcaggggtttacctttgatgcaggcccgacggttatcaccgatcccagtgccattgaagaactgtttgcactggcaggaaaacagttaaaagagtatgtcgaactgctgccggttacgccgttttaccgcctgtgttgggagtcagggaaggtctttaattacgataacgatcaaacccggctcgaagcgcagattcagcagtttaatccccgcgatgtcgaaggttatcgtcagtttctggactattcacgcgcggtgtttaaagaaggctatctgaagctcggtactgtcccttttttatcgttcagagacatgcttcgcgccgcacctcaactggcgaaactgcaggcatggagaagcgtttacagtaaggttgccagttacatcgaagatgaacatctgcgccaggcgttttctttccactcgctgttggtgggcggcaatcccttcgccacctcatccatttatacgttgatacacgcgctggagcgtgagtggggcgtctggtttccgcgtggcggcaccggcgcattagttcaggggatgataaagctgtttcaggatctgggtggcgaagtcgtgttaaacgccagagtcagccatatggaaacgacaggaaacaagattgaagccgtgcatttagaggacggtcgcaggttcctgacgcaagccgtcgcgtcaaatgcagatgtggttcatacctatcgcgacctgttaagccagcaccctgccgcggttaagcagtccaacaaactgcagactaagcgcatgagtaactctctgtttgtgctctattttggtttgaatcaccatcatgatcagctcgcgcatcacacggtttgtttcggcccgcgttaccgcgagctgattgacgaaatttttaatcatgatggcctcgcagaggacttctcactttatctgcacgcgccctgtgtcacggattcgtcactggcgcctgaaggttgcggcagttactatgtgttggcgccggtgccgcatttaggcaccgcgaacctcgactggacggttgaggggccaaaactacgcgaccgtatttttgcgtaccttgagcagcattacatgcctggcttacggagtcagctggtcacgcaccggatgtttacgccgtttgattttcgcgaccagcttaatgcctatcatggctcagccttttctgtggagcccgttcttacccagagcgcctggtttcggccgcataaccgcgataaaaccattactaatctctacctggtcggcgcaggcacgcatcccggcgcaggcattcctggcgtcatcggctcggcaaaagcgacagcaggtttgatgctggaggatctgatttgaggccatgcaggccgatccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggccttaataagcttcgatccgttaatcatggtgtaggcaacccaaataaaacaccaaaatatgcacaaggcagtttgttgtattctgtagtacagacaaaactaaaagtaatgaaagaagatgtggtgttagaaaaggaaacaatatcatgagtaatgtgtgagcattatgggaccacgaaataaaaagaacattttgatgagtcgtgtatcctcgatgagcctcaaaagttctctcaccccggataagaaacccttaagcaatgtgcaaagtttgcattctccactgacataatgcaaaataagatatcatcgatgacatagcaactcatgcatcatatcatgcctctctcaacctattcattcctactcatctacataagtatcttcagctaaatgttagaacataaacccataagtcacgtttgatgagtattaggcgtgacacatgacaaatcacagactcaagcaagataaagcaaaatgatgtgtacataaaactccagagctatatgtcatattgcaaaaagaggagagcttataagacaaggcatgactcacaaaaattcatttgcctttcgtgtcaaaaagaggagggctttacattatccatgtcatattgcaaaagaaagagagaaagaacaacacaatgctgcgtcaattatacatatctgtatgtccatcattattcatccacctttcgtgtaccacacttcatatatcatgagtcacttcatgtctggacattaacaaactctatcttaacatttagatgcaagagcctttatctcactataaatgcacgatgatttctcattgtttctcacaaaaagcattcagttcattagtcctacaacaactacactgaattcggcttcccaaatcgccgccaccatggccatcatactcgtacgagcagcgtcgccggggctctccgccgccgacagcatcagccaccaggggactctccagtgctccaccctgctcaagacgaagaggccggcggcgcggcggtggatgccctgctcgctccttggcctccacccgtgggaggctggccgtccctcccccgccgtctactccagcctgcccgtcaacccggcgggagaggccgtcgtctcgtccgagcagaaggtctacgacgtcgtgctcaagcaggccgcattgctcaaacgccagctgcgcacgccggtcctcgacgccaggccccaggacatggacatgccacgcaacgggctcaaggaagcctacgaccgctgcggcgagatctgtgaggagtatgccaagacgttttacctcggaactatgttgatgacagaggagcggcgccgcgccatatgggccatctatgtgtggtgtaggaggacagatgagcttgtagatgggccaaacgccaactacattacaccaacagctttggaccggtgggagaagagacttgaggatctgttcacgggacgtccttacgacatgcttgatgccgctctctctgataccatctcaaggttccccatagacattcagccattcagggacatgattgaagggatgaggagtgatcttaggaagacaaggtataacaacttcgacgagctctacatgtactgctactatgttgctggaactgtcgggttaatgagcgtacctgtgatgggcatcgcaaccgagtctaaagcaacaactgaaagcgtatacagtgctgccttggctctgggaattgcgaaccaactcacgaacatactccgggatgttggagaggatgctagaagaggaaggatatatttaccacaagatgagcttgcacaggcagggctctctgatgaggacatcttcaaaggggtcgtcacgaaccggtggagaaacttcatgaagaggcagatcaagagggccaggatgttttttgaggaggcagagagaggggtaactgagctctcacaggctagcagatggccagtatgggcttccctgttgttgtacaggcagatcctggatgagatcgaagccaacgactacaacaacttcacgaagagggcgtatgttggtaaagggaagaagttgctagcacttcctgtggcatatggaaaatcgctactgctcccatgttcattgagaaatggccagacctagggccatgcaggccgatccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgtgtttttatgattagagtcccgcaattatacatttaatacgcgatagaaaacaaaatatagcgcgcaaactaggataaattatcgcgcgcggtgtcatctatgttactagatcgtccagactagtaagggcgaattctagatgatggacattggaggaggttttgagcaatcgcacgactgctgtttggtttgctctggctttctgtttcgctgaatcttcatttctgatgaagggggtgatattaagttaaactgaattaattagactctctggtgggggaaatgaggtcaatcaacagcagattcaggcaactggcccgctattagtcttcagtctgcttatcgctattagtcttcagtctgcttatccttttgcgtgattgacatatcaccaactgtcagaaggggacacattacagatagctgtaataaatgatagataagatggaatcgaacagttggagtttcactgtgagactgcagctggttgtctcttttttcaatgtgtagtctggaagaacgtttctttcaactcctaatgtgccccttccagttatgtgagcgtgcgataagaaatcaagaagataactgttacattagaactgtctcacatttttttttcttttcatttacaccgaatcatggtgaacatgttcttctgcaacagtgcattggcatgcattatactaatcataacctaagtatttatctgatcacaagctttgtctaatcagatgagtaaaagatttgaaatccttagctgatcctccttgtaatctcaagtgtgatgttttcagtttcagatgaacagagcagtgatgtttatgttgctaacagtatgtgcaatactcattgacaggtgatgctgctgctagtagtatcagagctctacattgaaagattgcatctgattaggacggttcaggccagggtatgagccaaacctggtcttatctctcgagtctagagtgtgtggcgcgtgtgaattctgatggagtttactagagcggccgccaccgcggtggagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaattt

SEQ ID NO: 2 - DNA, 23 bp, Oryza sativa
gtggcgcgtgtgaattctgatgg

SEQ ID NO: 3 - DNA, 23 bp, Oryza sativa
gtggggcgtgtgaagtgtgaggg

SEQ ID NO: 4 - DNA, 25 bp, Oryza sativa
gtaccacgtgtctgaattctgatgg

SEQ ID NO: 5 - DNA, 23 bp, Oryza sativa
gtggcgtttctgaactctgatgg

SEQ ID NO: 6 - DNA, 23 bp, Oryza sativa
gtggcgtgcgggatttctgacgg

SEQ ID NO: 7 - DNA, 23 bp, Oryza sativa
gtggcccatatgagttctgaagg

SEQ ID NO: 8 - DNA, 23 bp, Oryza sativa
gtttcccttgtgaattctgatgg

SEQ ID NO: 9 - DNA, 23 bp, Oryza sativa
atggcccatgtgagttctgaagg

SEQ ID NO: 10 - DNA, 23 bp, Oryza sativa
gcggcgcgggtgaaagctgaggg

SEQ ID NO: 11 - DNA, 23 bp, Oryza sativa
gaggcgcgtgtggatcctgccgg

SEQ ID NO: 12 - DNA, 23 bp, Oryza sativa
gtgatgcgtgtgaatcgtgatgg

SEQ ID NO: 13 - DNA, 24 bp, Artificial Sequence
  Primer Target-A-gRNA-F
gtgtgcaaggcactcaactacgtg

SEQ ID NO: 14 - DNA, 24 bp, Artificial Sequence
  Primer Target-A-gRNA-R
aaaccacgtagttgagtgccttgc

SEQ ID NO: 15 - DNA, 24 bp, Artificial Sequence
  Primer Target-B-gRNA-F
gtgtgtggcgcgtgtgaattctga

SEQ ID NO: 16 - DNA, 24 bp, Artificial Sequence
  Primer Target-B-gRNA-R
aaactcagaattcacacgcgccac

SEQ ID NO: 17 - DNA, 24 bp, Artificial Sequence
  Primer Target-C-gRNA-F
gtgtgtagtggtagcagagctcag

SEQ ID NO: 18 - DNA, 24 bp, Artificial Sequence
  Primer Target-C-gRNA-R
aaacctgagctctgctaccactac

SEQ ID NO: 19 - DNA, 24 bp, Artificial Sequence
  Primer Target-D1-gRNA-F
gtgtgtggttttcgaacatacggt

SEQ ID NO: 20 - DNA, 24 bp, Artificial Sequence
  Primer Target-D1-gRNA-R
aaacaccgtatgttcgaaaaccac

SEQ ID NO: 21 - DNA, 24 bp, Artificial Sequence
  Primer Target-D2-gRNA-F
gtgtggtggtttacaaacatacga

SEQ ID NO: 22 - DNA, 24 bp, Artificial Sequence
  Primer Target-D2-gRNA-R
aaactcgtatgtttgtaaaccacc

SEQ ID NO: 23 - DNA, 24 bp, Artificial Sequence
  Primer Target-E1-gRNA-F
gtgtgcggggagaagaacgagact

SEQ ID NO: 24 - DNA, 24 bp, Artificial Sequence
  Primer Target-E1-gRNA-R
aaacagtctcgttcttctccccgc

SEQ ID NO: 25 - DNA, 24 bp, Artificial Sequence
  Primer Target-E2-gRNA-F
gtgtggaagacgccttcgacaggt

SEQ ID NO: 26 - DNA, 24 bp, Artificial Sequence
  Primer Target-E2-gRNA-R
aaacacctgtcgaaggcgtcttcc

SEQ ID NO: 27 - DNA, 18 bp, Artificial Sequence
  Primer Target-A-PCR-F
gcctgacagtgcgtggtc

SEQ ID NO: 28 - DNA, 20 bp, Artificial Sequence
  Primer Target-A-PCR-R
gcctcatcgctcctcgtgat

SEQ ID NO: 29 - DNA, 26 bp, Artificial Sequence
  Primer Target-B-PCR-F
gacagttggtgatatgtcaatcacgc

SEQ ID NO: 30 - DNA, 20 bp, Artificial Sequence
  Primer Target-B-PCR-R
tttgccgcttcgattcgtgt

SEQ ID NO: 31 - DNA, 23 bp, Artificial Sequence
  Primer Target-C-PCR-F
gaatagcagagtccacgagacga

SEQ ID NO: 32 - DNA, 22 bp, Artificial Sequence
  Primer Target-C-PCR-R
tttagagtacgtgggcacgtcg

SEQ ID NO: 33 - DNA, 26 bp, Artificial Sequence
  Primer Target-D-PCR-F
ttcggatgtgaacaatacactgctat

SEQ ID NO: 34 - DNA, 29 bp, Artificial Sequence
  Primer Target-D-PCR-R
acattagaatccatttccataattaaggg

SEQ ID NO: 35 - DNA, 19 bp, Artificial Sequence
  Primer Target-E-PCR-F
ggcgacggcaaacccgatg

SEQ ID NO: 36 - DNA, 19 bp, Artificial Sequence
  Primer Target-E-PCR-R
ggccacgcctcctgcacta

SEQ ID NO: 37 - DNA, 41 bp, Artificial Sequence
  Primer Cassette-AF
cggggtaccggtctcgctatcgttaatcatggtgtaggcaa

SEQ ID NO: 38 - DNA, 43 bp, Artificial Sequence
  Primer Cassette-AR
gctctagaggtctcacatgggttgttgtaggactaatgaactg

SEQ ID NO: 39 - DNA, 41 bp, Artificial Sequence
  Primer Cassette-CF
cggggtaccggtctcagatccgttaatcatggtgtaggcaa

SEQ ID NO: 40 - DNA, 43 bp, Artificial Sequence
  Primer Cassette-CR
gctctagaggtctcagtgtagttgttgtaggactaatgaactg

SEQ ID NO: 41 - DNA, 40 bp, Artificial Sequence
  Primer Cassette-BF
cggggtaccggtctcacatgagaattcggcttcccaaatc

SEQ ID NO: 42 - DNA, 40 bp, Artificial Sequence
  Primer Cassette-BR
gctctagaggtctcagatcgaagcttattaaggcccgatc

SEQ ID NO: 43 - DNA, 39 bp, Artificial Sequence
  Primer Cassette-DF
cggggtaccggtctctacactgaattcggcttcccaaat

SEQ ID NO: 44 - DNA, 44 bp, Artificial Sequence
  Primer Cassette-DR
gctctagaggtctctctggacgatctagtaacatagatgacacc

SEQ ID NO: 45 - DNA, 28 bp, Artificial Sequence
  Primer Target-B-PAM-F
gtgtgtggcgcgtgtgaattctgatgga

SEQ ID NO: 46 - DNA, 28 bp, Artificial Sequence
  Primer Target-B-PAM-R
aaactccatcagaattcacacgcgccac

SEQ ID NO: 47 - DNA, 28 bp, Artificial Sequence
  Primer Target-C-PAM-F
gtgtgtagtggtagcagagctcagagga

SEQ ID NO: 48 - DNA, 28 bp, Artificial Sequence
  Primer Target-C-PAM-R
aaactcctctgagctctgctaccactac

SEQ ID NO: 49 - DNA, 36 bp, Artificial Sequence
  Primer B-Left-Arm-F
cggggtaccctcgagacgtgcaatggagtgtaatac

SEQ ID NO: 50 - DNA, 29 bp, Artificial Sequence
  Primer B-Left-Arm-R
cggggtaccgaattcacacgcgccacaat

SEQ ID NO: 51 - DNA, 30 bp, Artificial Sequence
  Primer B-Right-Arm-F
tgctctagatgatggacattggaggaggtt

SEQ ID NO: 52 - DNA, 34 bp, Artificial Sequence
  Primer B-Right-Arm-R
tgctctagactcgagagataagaccaggtttggc

SEQ ID NO: 53 - DNA, 31 bp, Artificial Sequence
  Primer C-Left-Arm-F
cggggtaccgtcaggattggtaggaattagc

SEQ ID NO: 54 - DNA, 28 bp, Artificial Sequence
  Primer C-Left-Arm-R
cggggtaccagctctgctaccactagcc

SEQ ID NO: 55 - DNA, 32 bp, Artificial Sequence
  Primer C-Right-Arm-F
tgctctagagaggagtgtactgctacatgatg

SEQ ID NO: 56 - DNA, 31 bp, Artificial Sequence
  Primer C-Right-Arm-R
tgctctagaagaggtaggacgttacaggtgt

SEQ ID NO: 57 - DNA, 20 bp, Artificial Sequence
  Primer 1F
attggcaacgcactggattc

SEQ ID NO: 58 - DNA, 20 bp, Artificial Sequence
  Primer 2F
ggcagatcctggatgagatc

SEQ ID NO: 59 - DNA, 19 bp, Artificial Sequence
  Primer 1R
ggaagccgaattctcatgg

SEQ ID NO: 60 - DNA, 20 bp, Artificial Sequence
  Primer 3R
ctcatggaaggtgcacaacc

SEQ ID NO: 61 - DNA, 23 bp, Artificial Sequence
  Primer 3F
aggacggagaaagtactgcatag

SEQ ID NO: 62 - DNA, 21 bp, Artificial Sequence
  Primer 4R
aagaactccgaggttaaagcg

SEQ ID NO: 63 - DNA, 17 bp, Artificial Sequence
  Primer M13F
gtaaaacgacggccagt

SEQ ID NO: 64 - DNA, 23 bp, Artificial Sequence
  Primer Cas9p-Genotyping-F
cgagaacatcatccacctcttca

SEQ ID NO: 65 - DNA, 25 bp, Artificial Sequence
  Primer nos-Terminator-R
atctagtaacatagatgacaccgcg

SEQ ID NO: 66 - DNA, 73 bp, Artificial Sequence
  Primer pAcc-Engineer-F
agtgtgggtcttcgagaagacctgtttggtaccccgctagtctagagtgtggagacgattgcgtctctgttta

SEQ ID NO: 67 - DNA, 81 bp, Artificial Sequence
  Primer pAcc-Engineer-R
ctagtaaacagagacgcaatcgtctccacactctagactagcggggtaccaaacaggtcttctcgaagacccacactgtac

SEQ ID NO: 68 - DNA, 22 bp, Artificial Sequence
  Primer Chr5-insert-flanking-L
aataacagagaggctgagagtc

SEQ ID NO: 69 - DNA, 21 bp, Artificial Sequence
  Primer Chr5-insert-flanking-R
ggagaagcgtgggaataagaa

SEQ ID NO: 70 - DNA, 53 bp, Artificial Sequence
  End joining site with 4 basepair deletion of carotenoid cassette
gcgcgtgtgagaattcacacgcgccacacactctagactcgagagataagacc

SEQ ID NO: 71 - DNA, 43 bp, Artificial Sequence
  End joining site with no deletion of carotenoid cassette
cattgcacgtctcgagggtaccaaactccatcatgatggacat

SEQ ID NO: 72 - DNA, 22 bp, Artificial Sequence
  Guide RNA target A
caaggcactcaactacgtgagg

SEQ ID NO: 73 - DNA, 23 bp, Artificial Sequence
  Guide RNA target B
gtggcgcgtgtgaattctgatgg

SEQ ID NO: 74 - DNA, 22 bp, Artificial Sequence
  Guide RNA target C
ctactccggagtccggacaggg

SEQ ID NO: 75 - DNA, 22 bp, Artificial Sequence
  Guide RNA target D1
tggttttcgaacatacggtggg

SEQ ID NO: 76 - DNA, 23 bp, Artificial Sequence
  Guide RNA target D2
ggtggtttacaaacatacgatgg

SEQ ID NO: 77 - DNA, 22 bp, Artificial Sequence
  Guide RNA target E1
cggggagaagaacgagactcgg

SEQ ID NO: 78 - DNA, 23 bp, Artificial Sequence
  Guide RNA target E2
ggaagacgccttcgacaggtggg

SEQ ID NO: 79 - DNA, 64 bp, Artificial Sequence
  Junction of end joining site with 2 basepair deletion of carotenoid cassette
gcgaattgggtacagtgtgtggcgcgtgtgaattgatggacattggaggaggttttgagcaatc

SEQ ID NO: 80 - DNA, 136 bp, Artificial Sequence
  Junction of end joining site with 103 basepair insertion of carotenoid cassette
tggcgcgtgtgaattcgccgttggttgctcttcctctcccgtttgacgaaagccagcaaaaaaagctggaagttccttttaccctgctgtggcataaacggaacagccataatccgaagtgatggagtttggtacc

SEQ ID NO: 81 - DNA, 43 bp, Artificial Sequence
  Junction end joining site with 8 basepair deletion of carotenoid cassette
ggacagggctagtggtaagctctgctaccactacacactctag

SEQ ID NO: 82 - DNA, 43 bp, Artificial Sequence
  Junction end joining site with no deletion of carotenoid cassette
gacggtaccaaactcctctgatcagaggagtgtactgctacat